SEQUENCE LISTING (10 sequences)

SEQ ID NO: 1 - DNA, 2589 bp, Homo sapiens
  T-BET sequence
cggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttaaaaaaaaaaaaaaaaa

SEQ ID NO: 2 - DNA, 3674 bp, Homo sapiens
  EOMES sequence
aagtttccaagtggtcaacttgaccgatgctttggcaattgaaaaagggcagaaaggcgcgggctagtgggtggatggggacaaagatctaagtcaccttcttccagcgtgtgagcctgggaggagggtgggggtcctgaggagcaagaggtacgaggaaggaaaaggagagggcttctgggttagtttccacctcctgctttccaactcacggcgctttccttccggaaaggacgctggattcagggcgcgccagtacgcgcagtagcggcccgcgagtcggcaggtgggtagccccggcgcgggaggaaggggaagttaccttcccctcggaagagggcgctggctcccccatcctgcctttataataaggccaccggaggagaggaagcagccagctgccgtctgcgctttgcaaagcatgcagttaggggagcagctcttggtgagctcagtgaacctgcctggcgcgcacttctacccgctggagagtgcgcgaggcggcagcggcgggagcgctggccacctccccagcgcggccccctctcctcagaagttggacttagacaaagcgtccaagaagttttccggcagtctctcctgcgaggcggtgagcggggagcccgcagccgccagcgcaggggcccccgcggccatgcttagtgacaccgacgccggggacgcatttgccagcgctgcggcagtggccaagccggggcccccggacggccgcaagggctccccctgcggggaggaggagctgccctccgccgctgcagccgccgccgccgccgccgccgcggctgcggccactgcgcgctactccatggacagcctgagctccgagcggtactacctccagtcccccggtcctcaggggtcggagctggctgcgccctgctcactcttcccgtaccaggcggcggctggggcgccccacggacctgtgtacccggctcctaacggggcgcgctacccctacggctccatgctgccccccggcggcttccccgcggctgtgtgcccacccgggagggcgcagttcggcccaggagccggtgcgggcagtggcgcgggcggtagcagcggcgggggcggcggcccgggcacctatcagtacagccagggggctccgctctacgggccgtaccctggagccgcagcggcgggatcttgcggaggactggggggcctgggggttccaggttctggcttccgtgcccacgtctacctgtgcaaccggcctctgtggctcaaattccaccgccaccaaactgagatgatcattacgaaacagggcaggcgcatgtttcctttcttgagcttcaacataaacggactcaatcccactgcccactacaatgtgttcgtagaggtggtgctggcggaccccaaccactggcgcttccaggggggcaaatgggtgacctgtggcaaagccgacaataacatgcagggcaacaaaatgtatgttcacccagagtctcctaatactggttcccactggatgagacaggagatttcattcgggaaattaaaactcaccaataacaaaggcgcaaataacaacaacacccagatgatagtcttacaatccttacacaaataccaaccccgactgcatattgttgaagttacagaggatggcgtggaggacttgaatgagccctcaaagacccagacttttaccttctcagaaacgcaattcattgcagtgactgcctaccaaaacaccgatattactcaactaaagattgatcataacccctttgcaaaaggcttcagagacaactatgattccatgtacaccgcttcagaaaatgacaggttaactccatctcccacggattctcctagatcccatcagattgtccctggaggtcggtacggcgttcaatccttcttcccggagccctttgtcaacactttacctcaagcccgctattataatggcgagagaaccgtgccacagaccaacggcctcctttcaccccaacagagcgaagaggtggccaaccctccccagcggtggcttgtcacgcctgtccagcaacctgggaccaacaaactagacatcagttcctatgaatctgaatatacttctagcacattgctcccatatggcattaaatccttgccccttcagacatcccatgccctggggtattacccagacccaacctttcctgcaatggcagggtggggaggtcgaggttcttaccagaggaagatggcagctggactaccatggacctccagaacaagccccactgtgttctctgaagatcagctctccaaggagaaagtgaaagaggaaattggctcttcttggatagagacacccccttccatcaaatctctagattccaatgattcaggagtatacaccagtgcttgtaagcgaaggcggctgtctcctagcaactccagtaatgaaaattcaccctccataaagtgtgaggacattaatgctgaagagtatagtaaagacacctcaaaaggcatgggagggtattatgctttttacacaactccctaaagagttattttaacctcaaaaattagctaactttttgcagatggacttggtggtgttttttgttgtcttctttgcctaggttgccaaaaagatgtttgccttccaccttgatgcatcctgttttgtgcaattctctaaaagaaggtgccaaagctttttgattgctgcaggtaactgaaacaaacctagcatttttaaaaaataagattaatggaagactttaaggtattttaaaattcgaagggtatccaaggttctgtatttatttattggggagacactaacccttcaaagaagcaggctgtgaacattgggtgcccagtgctatcagatgagttaaaacctttgattctcatttctatttgtaaattcttaagcaaatagaagccgagtgttaaggtgttttgcttctgaaagagggctgtgccttccgtttcagaaggagacattttgctgttacattctgccaggggcaaaagatactaggcccaggagtcaagaaaagcttttgtgaaagtgatagtttcacctgactttgattccttaacccccggcttttggaacaagccatgtttgccctagtccaggattgcctcacttgagacttgctaggcctctgctgtgtgctggggtggccagtgggactcaggagagagcaagctaaggagtcaccaaaaaaaaaaaaaaaaaaaagggagaatttaaaagtgtacagttgtgtgtttagatacactatagaataatgtggtatatattgtacaaatagtctacataggtgtctgggataatgtaaaactggtgctttggctttgtaaagaatttgcaaatcacttaacagctgcaggggcaaggggagagtttcatcatccccatgatatttgggaatattctgtttacttcttagatagttaagaatgtattcagctactatgtactaacttgaaccgtgtttaaggaaaactcctatttcatcctcttcttgcgccatcccctctccctaacttggtaatgtgaagaaactaaaacctgataccacagctcctataggcattttagagatcttggatttttatgtacagtcttagtcatttttaataaatgtggttcagtaagggaacggaaaaaaaaaaaaaaa

SEQ ID NO: 3 - DNA, 37 bp, Artificial Sequence
  Fw-Tbet
aagttggatccaccatgggcatcgtggagccgggttg

SEQ ID NO: 4 - DNA, 52 bp, Artificial Sequence
  Rev-Tbet
aaagttctcgagtcagttgggaaaatagttataaaactgtccttcagcttcc

SEQ ID NO: 5 - DNA, 36 bp, Artificial Sequence
  Fw-Eomes
aaagttggatccaccatgcagttaggggagcagctc

SEQ ID NO: 6 - DNA, 42 bp, Artificial Sequence
  Rev-Eomes
aaagttctcgagttagggagttgtgtaaaaagcataataccc

SEQ ID NO: 7 - DNA, 38 bp, Artificial Sequence
  Fw-ToxEcoRI
atctcagaattcagtgaaatggacgtaagattttatcc

SEQ ID NO: 8 - DNA, 37 bp, Artificial Sequence
  Rev-ToxNgoMIV
aaagttgccggctcaagtaaggtacagtgctttgtcc

SEQ ID NO: 9 - DNA, 34 bp, Artificial Sequence
  Fw-Id2BamHI
ctatcaggatccgtcagcatgaaagccttcagtc

SEQ ID NO: 10 - DNA, 32 bp, Artificial Sequence
  Rev-Id2NgoMIV
aaagttgccggctcagccacacagtgctttgc

(19) United States
(12) Patent Application Publication
(10) Pub. No.: US 2022/0025327 A1
(43) Pub. Date: Jan. 27, 2022

(54) ACCELERATED HUMAN HEMATOPOEITIC STEM CELL DIFFERENTIATION TOWARDS MATURE NATURAL KILLER CELLS WITH ENHANCED ANTIBODY-DEPENDENT CYTOTOXIC ACTIVITY

(71) Applicant: Universiteit Gent, Gent (BE)

(72) Inventors: Laura Kiekens, Ressegem (BE); Georges Leclercq, Gent (BE)

(73) Assignee: Universiteit Gent, Gent (BE)

(21) Appl. No.: 17/281,840

(22) PCT Filed: Sep. 30, 2019
(86) PCT No.: PCT/EP2019/076459

§ 371 Date: Mar. 31, 2021

(30) Foreign Application Priority Data
Oct. 1, 2018 (EP) 18198021.0

(51) Int. Cl. C12N 5/0783; C12N 5/0789; C12N 15/86; A61K 35/14; A61K 35/17; C07K 14/47

(52) U.S. Cl. CPC C12N 5/0646; C12N 2502/1394; C12N 15/86; A61K 35/14; A61K 35/17; C07K 14/4702; C12N 2740/10043; C12N 2501/145; C12N 2501/125; C12N 2501/26; C12N 2501/2302; C12N 2501/2315; C12N 2501/2303; C12N 2501/2307; C12N 5/0647

(57) ABSTRACT

The present invention in general relates to a method of differentiating human hematopoietic stem cells (HSC) into mature natural killer (NK) cells; wherein said method is in particular characterized in that mature NK cells are obtainable very early during the differentiation method, and that these NK cells display increased CD16 expression and antibody-dependent cellular cytotoxicity (ADCC) (FIG. 11). The method of the invention specifically encompasses transfecting and/or transducing HSCs with at least one transcription factor selected from T-Box expressed in T cells (T-BET) and Eomesodermin (EOMES); or a combination thereof.

### FIELD OF THE INVENTION

The present invention in general relates to a method of differentiating hematopoietic stem cells (HSC) into mature natural killer (NK) cells; wherein said method is in particular characterized in that mature NK cells are obtainable very early during the differentiation method and, in addition, have enhanced antibody-dependent cellular cytotoxic (ADCC) activity (FIG. 11). The method of the invention specifically encompasses transfecting and/or transducing HSCs with at least one transcription factor selected from T-Box expressed in T cells (T-BET) and Eomesodermin (EOMES); or a combination thereof.

### BACKGROUND OF THE INVENTION

Innate lymphoid cells (ILC) are a novel lymphoid cell subfamily belonging to the innate immune system. The different ILC are developmentally related and characterized by a lymphoid morphology, the lack of gene-dependent rearrangement of antigen receptors and the absence of myeloid and dendritic phenotypical markers. Like helper T-cell subsets, ILC can be divided into three different groups according to distinct phenotypes, cytokine-secretion profiles and essential transcription factors [1,2].

Natural killer (NK) cells, which are considered as the prototypical ILC, are important cytotoxic cells [1]. They provide wide anti-tumor and anti-microbial protection upon activation by the release of cytolytic granules containing perforin and granzyme B. Besides cytotoxic effects, NK cells also contribute to immunomodulation by producing cytokines, including IFN-γ [3,4]. NK cells, like other lymphocytes, originate from CD34⁺ hematopoietic stem cells (HSC) in the bone marrow that differentiate through a common lymphoid progenitor stage. In secondary lymphoid tissues, human NK cell development is pursued whereby the cells sequentially develop into stage 1 (CD34⁺CD45RA⁺CD117⁻CD94⁻) pro-NK cells, followed by stage 2 or pre-NK cells (CD34⁺CD45RA⁺CD117⁺CD94⁻). Stage 1 and stage 2 cells are multipotent as they have T-cell, dendritic cell and NK cell developmental potential. Stage 3 cells (CD34⁻CD117⁺CD94⁻CD16⁻) are committed NK cell precursors since they can no longer develop into T-cells and dendritic cells. Stage 4 (CD34⁻CD56^{bright}CD94⁺CD16⁻) and stage 5 (CD34⁻CD56^{dim}CD94⁺CD16⁺) are mature NK cells [5,6]. Differentiation and maturation of NK cells and ILC is a complex molecular process tightly regulated by transcription factors. Many essential factors have been identified in the transcriptional control of murine ILC differentiation, thanks to the generation and availability of transcription factor-deficient mice [7]. In contrast to mice, the current knowledge on the role of transcription factors in human NK and ILC differentiation is extremely limited.

T-bet and Eomesodermin (Eomes) are two T-box transcription factors. T-bet is a protein encoded by the Tbx21 gene that is only expressed in hematopoietic cells. Eomes also plays an important role in vertebrate embryogenesis and shares homology with T-bet. T-bet is known as a master regulator essential for T-cell effector functions, including IFN-γ production and cytotoxicity. Moreover, T-bet and Eomes play a critical role in differentiation, maintenance and function of murine NK cells and ILC [8]. T-bet-deficient mice and Eomes^{flox/flox} Vav-Cre⁺ mice show decreased numbers of NK cells that mainly have an immature phenotype [9,11]. Mice lacking both T-bet and Eomes completely fail to develop NK cells [11]. These knockout mouse models show that both T-bet and Eomes are indispensable for NK cell development and terminal NK cell maturation. Furthermore, T-bet and Eomes are needed to maintain a mature NK cell phenotype, highlighted by the loss of maturity markers after induced deletion of T-bet/Eomes in mature NK cells [10]. Next to NK cells, particular subsets of ILC depend on T-bet and/or Eomes for their development. CD127⁺ ILC1 and natural cytotoxicity receptor (NCR)⁺ ILC3 express T-bet but lack Eomes. Eomes^{flox/flox} Vav-Cre⁺ mice have decreased numbers of NK cells but maintain ILC1. In contrast, T-bet-deficient mice have fewer NK cells, but completely lack ILC1. Mice lacking both T-bet and Eomes show a complete lack of ILC1. Also, no NCR⁺ ILC3 develop in the intestine of T-bet-deficient mice [9-12].

Because of their anti-tumor role, NK cells are abundantly researched as promising agents for cancer immunotherapy. Of the different NK cell-based therapeutic strategies one is the adoptive transfer of HSC into cancer patients. The other is to first differentiate HSC in vitro into mature NK cells that are then expanded to obtain sufficient NK cell numbers for transplantation. Whereas different approaches using NK cells in cancer therapy have already been used in the clinic, there still are some major limitations leading to relapse. Analysis of adoptively transferred mature NK cells in different murine tumor models revealed an exhaustion of the transferred NK cells, resulting in decreased cytotoxicity and IFN-γ production [13]. Importantly, this exhausted NK cell phenotype could be attributed to downregulation of the transcription factors T-BET and EOMES [13]. More recently, research proved that reduced T-BET and EOMES expression is also responsible for the NK cell functional impairment after HSC transplantation in leukemia patients. Reduction of T-BET and EOMES expression is already observed early after HSC transplantation. Downregulation of these transcription factors in NK cells is associated with increased nonrelapse mortality [14]. The role of thymocyte selection-associated HMG box protein (TOX) on the differentiation of human NK cells has been studied by Yun et al. [15] and in WO2012/046940. Vong et al. (2014) disclose that another member of the thymocyte selection-associated HMG box protein family, i.e. TOX2, is required in normal maturation of human NK cells and directly relates to T-BET expression [16]. CAR-T cells overexpressing T-BET are disclosed in WO2017/190100 and by Gacerez and Sentman [17].

Here, we reveal a method to accelerate human NK cell maturation from umbilical cord blood HSC in vitro, using retroviral constitutive overexpression constructs of either T-BET or EOMES. Whereas control transduced HSC require a culture period of 14 to 21 days to differentiate into mature functional NK cells, NK cells already appear on day 3 of culture with T-BET- or EOMES-transduced HSC. These early arising NK cells have a fully mature phenotype and are also highly functional regarding specific cytotoxicity and IFN-γ production. Importantly, the NK cells also display enhanced ADCC activity. This accelerated NK cell differentiation and maturation of NK cells with enhanced ADCC activity upon T-BET or EOMES transduction of HSC can provide a novel tool to optimize the NK cell-based adoptive cell therapies.

### SUMMARY OF THE INVENTION

In a first aspect, the present invention provides an ex vivo method of differentiating hematopoietic stem cells (HSC) into mature natural killer (NK) cells, said method comprising the steps of:
a) providing isolated HSCs;
b) culturing said cells of step a) in medium containing thrombopoietin (TPO), stem cell factor (SCF) and FMS-like tyrosine kinase 3 ligand (FLT3-L);
c) transfecting and/or transducing said cells of step b) with at least one transcription factor selected from the list comprising: T-Box expressed in T cells (T-BET) or Eomesodermin (EOMES); or a combination thereof;
d) culturing the cells obtained from step c) in a medium containing at least one cytokine selected from the list comprising IL-2 or IL-15; preferably IL-15;

whereby said mature NK cells are obtainable from day 3, in particular from day 4 or 5, after the start of step d).

In a specific embodiment of the present invention, said mature NK cells are at least of stage 4, in particular at stage 4 and stage 5 NK cells. At least from 5 days after transfection or transduction, stage 4 NK cells are present and/or can be obtained. At least from 9 days after transfection or transduction, stage 5 NK cells are present and/or can be obtained.

In another particular embodiment, said medium of step b) is complete Iscove's Modified Dulbecco's Medium (IMDM medium), in particular comprising about 1 to 20% fetal calf serum (FCS).

In yet a further embodiment of the present invention, said TPO is present at a concentration from about 1 ng/ml to about 100 ng/ml; preferably about 20 ng/ml.

In a still further embodiment, said SCF is present at a concentration from about 5 ng/ml to about 500 ng/ml; preferably about 100 ng/ml.

In another embodiment, said FLT3-L is present at a concentration from about 5 ng/ml to about 500 ng/ml; preferably about 100 ng/ml.

In yet a further embodiment of the invention, said medium of step d) further comprises a cytokine selected from the list comprising FLT3-L, SCF, IL-3 or IL-7.

In another particular embodiment, said IL-2 and/or IL-15 is present at a concentration from about 0.5 ng/ml to about 50 ng/ml; preferably about 10 ng/ml.

In a further embodiment, step d) of the method of the present invention is a co-culturing step using an (inactivated) feeder cell line, in particular a stromal cell line, such as e.g. using EL08.1D2 cells or OP9 cells.

In a further embodiment of the method of the present invention, in step c) said cells are transduced with a (retroviral) vector comprising a nucleic acid encoding said at least one transcription factor.

In a further aspect, the present invention provides HSC cells which are characterized in that they have been transfected and/or transduced with at least one transcription factor selected from the list comprising: T-Box expressed in T cells (T-BET), Eomesodermin (EOMES) or a combination of T-BET and EOMES.

The present invention also provides differentiated NK cells obtained using the method according to this invention; more in particular differentiated NK cells whereby CD16 expression of said NK cells is increased compared to non-transfected or non-transduced control cells, or to control transfected or control transduced cells.

The present invention also provides the differentiated NK cells as disclosed herein for use in inducing antibody-dependent cellular cytotoxicity in a subject having cancer.

### BRIEF DESCRIPTION OF THE DRAWINGS

FIG. 1. Overexpression of T-BET/EOMES in HSC leads to a strong decrease in NK cell progenitors. Upon transduction with T-BET, EOMES or control overexpression vectors in cord blood-derived HSC, CD34⁺Lin⁻eGFP⁺ precursor cells were sorted and cultured in the NK cell/ILC3 differentiation culture in the presence of EL08-1D2 stromal cells. (A) Expression of T-BET and EOMES was determined on the indicated time points in the T-BET and EOMES overexpression and in the control (ctrl) cultures with flow cytometry. Representative histograms for eGFP⁺CD45⁺ gated cells are shown (dotted line=fluorescence minus one (FMO) control). The mean fluorescence intensity (MFI) of T-BET and EOMES expression is presented in the bar charts as mean±SEM (n=5-9). (B) Control and overexpression cultures were analyzed on day 3 by flow cytometry to evaluate the presence of the NK cell precursors in eGFP⁺CD45⁺ gated cells, including HSC (CD34⁺CD45RA⁻), stage 1 (CD34⁺CD45RA⁺CD117⁻), stage 2 (CD34⁺CD45RA⁺CD117⁺) cells, and stage 3 (CD34⁻CD117⁺CD94⁻) cells. Representative dot plots are shown. Arrows indicate the gating strategy. The numbers in the gates indicate the percentage. Absolute cell numbers of the different NK cell stages are shown in the bar chart as mean±SEM (n=5-6). * and ** represent a p-value of <0.05 and <0.01, respectively.

FIG. 2. Accelerated human NK cell development upon T-BET and EOMES overexpression in HSC. Upon transduction with T-BET, EOMES or control overexpression vectors in cord blood-derived HSC, CD34⁺Lin⁻eGFP⁺ precursor cells were sorted and cultured in the NK cell/ILC3 differentiation culture in the presence of EL08-1D2 stromal cells. Cultures were analyzed on the indicated time points by flow cytometry. (A) Representative dot plots of eGFP⁺CD45⁺CD11a⁺ gated cells are shown. Cells in the upper right quadrant represent the total mature NK cell population (CD56⁺CD94⁺). The numbers indicate the percentages. (B) Representative dot plots of gated total NK cells (eGFP⁺CD45⁺CD11a⁺CD56⁺CD94⁺) are shown, in which stage 4 (CD56⁺CD16⁻) and stage 5 (CD56⁺CD16⁺) NK cells can be discriminated. (C) Absolute cell numbers of stage 4 and stage 5 NK cells at different time points are indicated as mean±SEM (n=6-10). (D) Percentages of stage 5 NK cells (CD56⁺CD16⁺) of the total NK cell population (eGFP⁺CD45⁺CD11a⁺CD56⁺CD94⁺) at different time points are indicated as mean±SEM (n=6-9). * and ** represent a p-value of <0.05 and <0.01, respectively.

FIG. 3. Human NK cell development upon T-BET and EOMES overexpression is also accelerated in the absence of stromal feeder cells. Upon transduction with T-BET, EOMES or control overexpression vectors in cord blood-derived HSC, CD34⁺Lin⁻eGFP⁺ precursor cells were sorted and cultured in the NK cell/ILC3 differentiation culture in the absence of EL08-1D2 stromal cells. Cultures were analyzed on the indicated time points by flow cytometry. (A) Representative dot plots of eGFP⁺CD45⁺CD11a⁺ gated cells are shown. Cells in the upper right quadrant represent the total mature NK cell population (CD56⁺CD94⁺). The numbers indicate the percentages. (B) Representative dot plots of gated total NK cells (eGFP⁺CD45⁺CD11a⁺CD56⁺CD94⁺) are shown, in which stage 4 (CD56⁺CD16⁻) and stage 5 (CD56⁺CD16⁺) NK cells can be discriminated. (C) Absolute cell numbers of stage 4 and stage 5 NK cells at different time points are indicated as mean±SEM (n=2).

FIG. 4. NK cell differentiation upon T-BET and EOMES overexpression in HSC remains dependent on IL-15. Upon transduction with T-BET, EOMES or control overexpression vectors in cord blood-derived HSC, CD34⁺Lin⁻eGFP⁺ precursor cells were sorted and cultured in the NK cell/ILC3 differentiation culture, in the presence of EL08-1D2 stromal cells, whereby IL-15 was not included in the cytokine mix. Cultures were analyzed on the indicated time points by flow cytometry. Representative dot plots of eGFP⁺CD45⁺CD11a⁺ gated cells with the percentages indicated in each quadrant. No NK cells (CD56⁺CD94⁺) develop upon T-BET and EOMES overexpression in HSC in the absence of IL-15.

FIG. 5. T-BET and EOMES overexpression in HSC inhibits ILC3 differentiation and does not induce T, NKT or B cell differentiation. Upon transduction with T-BET, EOMES or control overexpression vectors in cord blood-derived HSC, CD34⁺Lin⁻eGFP⁺ precursor cells were sorted and cultured in the NK cell/ILC3 differentiation culture. Cultures were analyzed on the indicated time points by flow cytometry. (A) Representative dot plots of eGFP⁺CD45⁺CD11a⁻CD94⁻CD117⁺ gated cells are shown. Cells in the upper right quadrant represent ILC3 cells (NKp44⁺ RORγt⁺). The bar charts represent the absolute cell numbers of ILC3 as mean±SEM (n=6-7). * represents a p-value <0.05. (B) The presence of the other lymphocyte populations was determined on day 14 in both T-BET and EOMES overexpression cultures and control cultures. Representative dot plots of eGFP⁺CD45⁺ gated cells are shown for T cells (CD56⁻CD3⁺), NKT cells (CD56⁺CD3⁺) and B cells (CD56⁻CD19⁺). The numbers indicate the corresponding percentages.

FIG. 6. NK cells developing upon T-BET/EOMES overexpression in HSC are phenotypically and morphologically mature. To further characterize the NK cells differentiating upon T-BET and EOMES overexpression in HSC, flow cytometry was used to determine the expression of different mature NK cell markers. (A) Representative histograms showing the expression of the indicated NK cell markers by T-BET or EOMES overexpressing NK cells on day 3 and day 7 and by control transduced NK cells on day 14. (B) The MFI or the percentage of the indicated NK cell markers is presented as mean±SEM (n=5-9). *, ** or *** represent a p-value <0.05, <0.01 or <0.001, respectively. (C) NK cells were sorted on day 3 and day 7 of T-BET and EOMES overexpression cultures, and on day 19 of control cultures. Sorted cells were stained with Wright-Giemsa and microscopically analyzed. The arrows indicate the cytotoxic granules in the cytoplasm. The percentage of granulated NK cells is shown in the bar chart.

FIG. 7. NK cells generated upon T-BET/EOMES overexpression in HSC are functionally mature. (A) Day 21 T-BET and EOMES overexpressing NK cells and control NK cells were sorted and co-cultured with ⁵¹Cr-labeled K562 target cells for 4 h. The percentage of specific lysis at different effector:target (E:T) ratios is shown (mean±SEM) (n=6-9). (B) Cells from both T-BET/EOMES overexpression and control cultures on day 21 were stimulated with K562 target cells for 2 h, whereafter CD107a expression in gated NK cells was determined by flow cytometry. The percentage of CD107a expression in NK cells is shown in the bar chart as mean±SEM (n=9). (C) Cells from day 21 T-BET and EOMES overexpression cultures and control cultures were stimulated with PMA/ionomycin, IL-12/IL-18 or IL-12/IL-18/IL-15 for 24 h, whereafter IFN-γ and TNF-α production was measured by flow cytometry in gated NK cells. The percentages of IFN-γ and TNF-α producing NK cells are shown as mean±SEM (n=6) in the bar chart. (B-C) * represents a p-value <0.05.

FIG. 8. NK cells developing upon EOMES overexpression in HSC display increased antibody-dependent cellular cytotoxic (ADCC) activity. (A) T-BET and EOMES overexpressing NK cells and control NK cells from day 21 cultures were sorted and co-cultured with ⁵¹Cr-labeled Raji target cells in the presence or absence of Rituximab (RTX). The percentage of specific lysis as a function of different E:T ratios is shown in the bar charts as mean±SEM (n=15). (B) Cells from both T-BET/EOMES overexpression cultures and control cultures from day 21 were stimulated for 2 h with K562, or with Raji target cells in the presence or absence of RTX. The percentage of CD107a expression in gated NK cells was determined by flow cytometry and is shown in the bar charts as mean±SEM (n=6). (A-B) *, ** or *** represent a p-value <0.05, <0.01 or <0.001, respectively.

FIG. 9. T-BET and EOMES overexpression affects the transcriptome of HSC. HSC (CD34⁺Lin⁻eGFP⁺) were sorted on day 0 from T-BET and EOMES overexpression and control cultures and RNA sequencing was performed (n=5). (A) Volcano plots show gene expression in HSC from T-BET or EOMES overexpression cultures versus control cultures. Blue- and red-colored dots represent transcripts that are significantly down- or up-regulated (FDR <0.05), respectively. Selected differentially expressed transcription factors are indicated. (B) GSEA analysis was performed on the differentially expressed genes in HSC from T-BET or EOMES overexpression versus control cultures using the top 500 genes expressed in an NO″ cell-specific gene set. NES=normalized enrichment score.

FIG. 10. ID2, TOX and ETS-1 overexpression in HSC does not accelerate human NK cell differentiation. ID2, TOX, ETS-1 or the control vector were transduced in cord blood-derived HSC. CD34⁺Lin⁻eGFP⁺ precursor cells were sorted and cultured in the NK cell/ILC3 differentiation culture. Cultures were analyzed on the indicated time points by flow cytometry. (A) Absolute cell numbers (mean±SEM) of the indicated cell populations are shown for ID2 and TOX overexpression cultures and compared to control-cultures. (B) Absolute cell numbers (mean±SEM) of the NK cells for the indicated conditions. p27 is a dominant-negative isoform that inhibits signaling of endogenous ETS-1, whereas p51 is the full length isoform. * represents a p-value <0.05

FIG. 11. Graphical abstract

Cord blood-derived HSC are transduced with cDNA encoding the human transcription factors T-BET or EOMES and are cultured in vitro in the NK cell differentiation culture. T-BET and EOMES overexpression in HSC leads to a drastic acceleration of NK cell maturation and the NK cells display increased CD16 (FcγRIII)-expression and antibody-dependent cellular cytotoxicity (ADCC).

### DETAILED DESCRIPTION OF THE INVENTION

As used herein, the singular forms “a”, “an”, and “the” include both singular and plural referents unless the context clearly dictates otherwise. The terms “comprising”, “comprises” and “comprised of” as used herein are synonymous with “including”, “includes” or “containing”, “contains”, and are inclusive or open-ended and do not exclude additional, non-recited members, elements or method steps. The term “about” as used herein when referring to a measurable value such as a parameter, an amount, a temporal duration, and the like, is meant to encompass variations of +/−20% or less, preferably +1-10% or less, more preferably +/−5% or less, of and from the specified value, insofar such variations are appropriate to perform in the disclosed invention. It is to be understood that the value to which the modifier “about” refers is itself also specifically, and preferably, disclosed. Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any >3, >4, >5, >6 or >7 etc. of said members, and up to all said members. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In a first aspect, the present invention provides a method of differentiating hematopoietic stem cells (HSC) into mature natural killer (NK) cells, said method comprising the steps of:
a) providing isolated HSCs;
b) culturing said cells of step a) in medium containing thrombopoietin (TPO), stem cell factor (SCF) and FMS-like tyrosine kinase 3 ligand (FLT3-L);
c) transfecting and/or transducing said cells of step b) with at least one transcription factor selected from the list comprising: T-Box expressed in T cells (T-BET) or Eomesodermin (EOMES); or a combination thereof;
d) culturing the cells obtained from step c) in a medium containing at least one cytokine selected from the list comprising IL-2 or IL-15; preferably IL-15;

whereby said mature NK cells are obtainable from day 3 after the start of step d).

In a further aspect, the present invention provides a method of differentiating hematopoietic stem cells (HSC) into mature natural killer (NK) cells, said method comprising the steps of:
a) providing isolated HSCs;
b) culturing said cells of step a) in medium containing thrombopoietin (TPO), stem cell factor (SCF) and FMS-like tyrosine kinase 3 ligand (FLT3-L);
c) transfecting and/or transducing said cells of step b) with at least one transcription factor selected from the list comprising: T-Box expressed in T cells (T-BET) or Eomesodermin (EOMES); or a combination thereof;
d) culturing the cells obtained from step c) in a medium containing FLT3L, SCF, IL-3 and IL-7, further comprising at least one cytokine selected from the list comprising IL-2 or IL-15; preferably IL-15; whereby said mature NK cells are obtainable from day 3 after the start of step d).

In a specific embodiment, human HSC are purified from cord blood and precultured for 2 days in the presence of FLT3L, SCF and TPO to induce proliferation, which enhances the transduction efficiency. Thereafter, cells are transduced with retroviral supernatant of the LZRS virus, containing the encoding cDNA of either TBET and EOMES. The viral construct also contains the EGFP reporter gene, that enables selection of the transduced cells by flow cytometric sorting 1-2 days after transduction. The retroviral transduction results in integration in the DNA of the host cell and in constitutive overexpression (significant) higher expression of the encoded protein as compared to the control transduced cells (displayed as mean fluorescence intensity (MFI)); or when the basal level of protein expression is exceeded) of the encoded protein, as measured by flow cytometric analysis. The negative control vector only contains EGFP. The transduced cells are then cultured on the EL08-1D2 stromal cell line, in the presence of FLT3L, SCF, IL-3, IL-7 and IL-15. In this condition, NK cell differentiation starting from HSC is enabled.

In the context of the present invention, the term “hematopoietic stem cells (HSCs)” is meant to be stem cells which give rise to blood cells during the process called hematopoiesis. The HSC of the present invention may be obtained/isolated from any suitable sample, such as for example from umbilical cord blood as further described in the examples part, or alternatively from placenta, placental blood, placental perfusate, peripheral blood, bone marrow, thymus, spleen, or liver. Enrichment of the cell population for HSCs may for example be done by cell sorting on the basis of CD34 expression, since CD34 is known to be a HSC marker. Hematopoietic cells used in the methods provided herein can be obtained from a single individual, e.g., from a single placenta, or from a plurality of individuals, e.g., can be pooled. Where the hematopoietic cells are obtained/isolated from a plurality of individuals and pooled, the hematopoietic cells may be obtained from the same tissue source. Thus, in various embodiments, the pooled hematopoietic cells are all from placenta, e.g., placental perfusate, all from placental blood, all from umbilical cord blood, all from peripheral blood, and the like.

In the context of the present invention, the term “differentiating” is meant to be a process in which a cell changes from one cell type to another. By using the method of the present invention, HSCs can be changed into mature natural killer cells, during such differentiation process. Specifically, production of NK cells by the present method comprises expanding a population of hematopoietic stem cells. During cell expansion, a plurality of hematopoietic stem cells within the hematopoietic cell population differentiate into NK cells.

“Natural killer cells” or “NK cells” are a type of cytotoxic lymphocytes which are critical to the innate immune system. In the human body, NK cells for example provide rapid response to viral-infected cells, and respond to tumor formation. Differentiation of NK cells in vitro is a complex process regulated by transcription factors, and often a very time consuming process as well. In addition, CD16 expression of in vitro differentiated NK cells is relatively low, resulting in low antibody-dependent cellular cytotoxic (ADCC) capacity. The method of the present invention provides a solution to these problems in that mature NK cells can be obtained much more rapidly compared to prior art known differentiation methods (e.g. after about 3-7 days vs 14-21 days of culture; in particular after 3, 4, 5, 6 or 7 days of culture; or in the alternative after 5, 6, 7, 8 or 9 days after transfection or transduction of the cells e.g. as in step (c) described herein). In addition, the thus obtained NK cells display about 2-10 fold, in particular about 2-5 fold, more in particular about 2.5- to 4.5-fold higher CD16 expression (as compared to control cells), resulting in increased ADCC activity. The thus obtained NK cells are thus highly suitable in human medicine, such as in anti-cancer therapy or NK cell-based adoptive cell therapies.

Hence, the present invention also provides differentiated NK cells whereby CD16 expression of said NK cells is increased compared to non-transfected or non-transduced control cells, or to control transfected or control transduced cells. The present invention also provides differentiated NK cells as defined herein, for use in inducing antibody-dependent cellular cytotoxicity in a subject having cancer.

In the context of the present invention, “thrombopoetin (TPO)” is a protein, which is also known as megakaryocyte growth and development factor. In the human body, it is produced by the liver and kidney and regulates the production of platelets. In a specific embodiment of the present invention, said TPO is present in the medium (such as e.g. used in step b) at a concentration from about 1 ng/ml to about 100 ng/ml; more specifically, from about 5 ng/ml to about 50 ng/ml; more in particular from about 10 ng/ml to about 30 ng/ml; in particular about 15 ng/ml, about 20 ng/ml or about 25 ng/ml.

In the context of the present invention, “stem cell factor (SCF)”, also known as KIT-ligand, is a cytokine that plays an important role in hematopoiesis. In the present context, SCF contributes to self-renewal and maintenance of HSCs. In a specific embodiment of the present invention, said SCF is present in the medium (such as e.g. used in step b) at a concentration from about 5 ng/ml to about 500 ng/ml; more specifically, from about 50 ng/ml to about 200 ng/ml; more in particular from about 90 ng/ml to about 110 ng/ml; in particular about 90 ng/ml, about 100 ng/ml or about 110 ng/ml. SCF may also be used as an additional interleukin in the medium used in the culturing step d), where it may then be present at a concentration from about 1 ng/ml to about 100 ng/ml; more specifically, from about 5 ng/ml to about 50 ng/ml; more in particular from about 10 ng/ml to about 30 ng/ml; in particular about 15 ng/ml, about 20 ng/ml or about 25 ng/ml.

In the context of the present invention, FLT3-ligand (FLT-3-L), also known as FMS-like tyrosine kinase 3 ligand, is an endogenous small molecule that functions as a cytokine and growth factor that increases the number of immune cells by activating the hematopoietic progenitors. In a specific embodiment of the present invention, said FLT3-L is present in the medium (such as e.g. used in step b) at a concentration from about 5 ng/ml to about 500 ng/ml; more specifically, from about 50 ng/ml to about 200 ng/ml; more in particular from about 90 ng/ml to about 110 ng/ml; in particular about 90 ng/ml, about 100 ng/ml or about 110 ng/ml. FLT3-L may also be used as an additional interleukin in the medium used in the culturing step d), where it may then be present at a concentration from about 1 ng/ml to about 50 ng/ml; more specifically, from about 5 ng/ml to about 25 ng/ml; more in particular from about 5 ng/ml to about 15 ng/ml; in particular about 5 ng/ml, about 10 ng/ml or about 15 ng/ml.

In the context of the present invention, the term “transfecting or transfection” is meant to be a process for deliberately introducing naked or purified nucleic acids, such as vectors (DNA or RNA) or mRNA molecules into eukaryotic cells. The term “transducing or transduction” is meant to be a type of transfection process using virus-mediated gene transfer, e.g. by using a retroviral or lentiviral vector. In the context of the present invention, any suitable method for transfection/transduction of HSC cells may be used, such as electroporation, calcium phosphate transfection or RetroNectin-mediated transduction, as further detailed in the examples herein after.

Key to the current invention, is the transfection or transduction of T-BET and/or Eomesodermin (EOMES) transcription factors in HSCs, which leads to a significant reduction in time of the differentiation process into mature NK cells, and which also leads to increased CD16 expression in the thus obtained NK cells, resulting in increased ADCC.

T-BET (or ‘T-Box expressed in T cells’) is a transcription factor involved in the regulation of developmental processes, more specifically it regulates the development of naive T lymphocytes. As detailed in the examples part, it was surprisingly found that overexpression of T-BET in HSCs (after transfection/transduction) resulted in a significant increase in the absolute number of mature stage 4 and stage 5 NK cells already 3 days after the culturing step d). Human T-BET protein and nucleic acid sequences included herein are any homolog or artificial sequence that is substantially identical, i.e. at least 80%, 85%, 87%, 89%, 90%, 91%, 92%, 93%, 94%, 95%, 96%, 97%, 98%, 99% or 100% identical to the corresponding T-BET sequence identified by NCBI Accession number NM_013351.1 (incorporated herein by reference) (SEQ ID NO:1 for the nucleic acid sequence). T-BET as used herein encompasses also natural variants of the aforementioned specific T-BET protein. Such variants have at least the same essential biological and immunological properties as the specific T-BET protein.

EOMES (or eomesodermin) is a transcription factor involved in the regulation of developmental processes of vertebrates, more specifically it controls regulation of neural stem cells as well as other related cells. As detailed in the examples part, it was surprisingly found that overexpression of EOMES (after transfection/transduction) resulted in a significant increase in the absolute number of mature stage 4 and stage 5 NK cells already 3 days after the culturing step d). In addition, the thus obtained NK cells displayed increased ADCC activity.

In said context, the NK cells of the present invention, and more specific the NK cells overexpressing EOMES, are of particular interest for use in combination with therapeutic antibodies. NK cell (adoptive) therapy can thus be combined with injection of a monoclonal antibody specifically recognizing a tumor antigen. Such antibodies are often used in cancer immunotherapy. By combining NK cells and tumor antigen-specific antibody therapies, the tumor cells are efficiently targeted by the NK cells, leading to a better outcome.

In one embodiment, the invention provides the mature NK cells of the invention, characterized by high expression of CD16, in combination with an antibody, in particular a monoclonal antibody. The enhanced expression of CD16 of ex vivo differentiated NK cells might be utilized in therapeutic settings combining the cytotoxic activity of NK cells with therapeutic antibodies against e.g. malignant cells.

Human EOMES protein and nucleic acid sequences included herein are any homolog or artificial sequence that is substantially identical, i.e. at least 80%, 85%, 87%, 89%, 90%, 91%, 92%, 93%, 94%, 95%, 96%, 97%, 98%, 99% or 100% identical to the corresponding EOMES sequence identified by NCBI Accession number NM_001278182.1 (incorporated herein by reference) (SEQ ID NO:2 for the nucleic acid sequence). EOMES as used herein encompasses also natural variants of the aforementioned specific EOMES protein. Such variants have at least the same essential biological and immunological properties as the specific EOMES protein.

The present invention not only discloses the use of a single transcription factor selected from T-BET or EOMES, it also encompasses the combined use of both transcription factors. As the gene targets of T-BET and EOMES are (partially) different, it may be advantageous to combine both transcription factors as this might result in a synergistic effect.

The transcription factors of the present invention may be transfected/transduced in the cells as provided herein using any suitable method. The methods used for transfection or transduction are generally known to the skilled person and are not limiting to the present invention. In a specific embodiment, said cells are transduced with a viral vector, in particular a retroviral vector, comprising a nucleic acid encoding said at least one transcription factor. Alternatively, said cord blood HSC can be transfected with mRNA encoding these transcription factors. This will result in transient TBET and EOMES protein transcription, which, given the relatively short half-life of mRNA, will be lost after a short period of time.

Another approach is to generate an inducible retroviral vector, such as by using a construct generating a fusion protein between the transcription factor of interest and a mutant estrogen receptor (ERT2) in the retroviral vector (e.g. LZRS). The fusion protein is followed by a 2A-sequence and the enhanced green fluorescent protein (eGFP) reporter gene, which allows discrimination of transduced from untransduced cells. Upon retroviral transduction, CD34⁺Lineage⁻(CD3/14/19/56) eGFP⁺cord blood HSC can be sorted and put in differentiation culture to study the impact of the transduced transcription factor on NK cell development. The transduced transcription factor/ERT2 fusion protein is constitutively expressed but it remains cytosolic, and thus inactive, by binding to heat shock proteins. The addition of tamoxifen dissociates the heat shock proteins, translocates the transcription factor to the nucleus, and thus activates the transcription factor. Transcription factors can be activated from the start of the culture and this activation can be stopped thereafter at any time point by removing tamoxifen from the culture medium.

After the transcription/transduction step, the cells obtained therefrom are cultured in a medium containing at least one cytokine. In a particular embodiment, said cytokine is interleukin-3 (IL-3), interleukine-7 (IL-7), interleukin-2 (IL-2) and/or interleukin-15 (IL-15). In a preferred embodiment, the at least one cytokine is IL-15.

In the context of the present invention, “interleukin-3” (IL-3) is an interleukin that stimulates differentiation of HSC towards myeloid precursors. In addition to IL-7, it stimulates the differentiation of HSC towards lymphoid precursors. In a specific embodiment of the present invention, said IL-3 is present in the medium (such as e.g. of step d) at a concentration from about 5 ng/ml to about 500 ng/ml; more specifically, from about 0.5 ng/ml to about 50 ng/ml; more in particular from about 1 ng/ml to about 20 ng/ml; in particular about 5 ng/ml, about 10 ng/ml or about 15 ng/ml; alternatively from about 0.5 ng/ml to about 25 ng/ml; more specifically, from about 1 ng/ml to about 15 ng/ml; more in particular from about 1 ng/ml to about 10 ng/ml; in particular about 10 ng/ml, about 5 ng/ml or about 15 ng/ml.

In the context of the present invention, “interleukin-7” (IL-7) is an interleukin that stimulates differentiation of HSC towards lymphoid precursors. Furthermore, IL-7 plays an important role in regulating survival and expansion of mature NK cells. In a specific embodiment of the present invention, said IL-7 is present in the medium (such as e.g. of step d) at a concentration from about 5 ng/ml to about 500 ng/ml; more specifically, from about 0.5 ng/ml to about 50 ng/ml; more in particular from about 1 ng/ml to about 20 ng/ml; in particular about 5 ng/ml, about 10 ng/ml or about 15 ng/ml; alternatively from about 1 ng/ml to about 100 ng/ml; more specifically, from about 5 ng/ml to about 50 ng/ml; more in particular from about 10 ng/ml to about 30 ng/ml; in particular about 15 ng/ml, about 20 ng/ml or about 25 ng/ml.

In the context of the present invention, “IL-2” or “interleukin-2” is a type of cytokine signaling molecule in the immune system which regulates the activities of white blood cells that are responsible for immunity, in forming part of the body's natural response against microbial infections. In a specific embodiment of the present invention, said IL-2 is present in the medium (such as e.g. of step d) at a concentration from about 5 ng/ml to about 500 ng/ml; more specifically, from about 0.5 ng/ml to about 50 ng/ml; more in particular from about 1 ng/ml to about 20 ng/ml; in particular about 5 ng/ml, about 10 ng/ml or about 15 ng/ml.

In the context of the present invention, “IL-15” or “interleukin-15” is a type of cytokine with structural similarity to IL-2. IL-15 is secreted by mononuclear phagocytes following infection by viruses and it induces cell proliferation of natural killer cells. In a specific embodiment of the present invention, said IL-15 is present in the medium (such as e.g. of step d) at a concentration from about 5 ng/ml to about 500 ng/ml; more specifically, from about 0.5 ng/ml to about 50 ng/ml; more in particular from about 1 ng/ml to about 20 ng/ml; in particular about 5 ng/ml, about 10 ng/ml or about 15 ng/ml; alternatively from about 1 ng/ml to about 50 ng/ml; more specifically, from about 5 ng/ml to about 25 ng/ml; more in particular from about 5 ng/ml to about 15 ng/ml; in particular about 5 ng/ml, about 10 ng/ml or about 15 ng/ml.

In the context of the present invention, the stage (e.g. maturity) of the NK cells of the present invention is determined by evaluation of phenotypic NK cell markers (CD56, CD94, CD16) present on the cell surface of the NK cells by methods generally known, in particular by means of flow cytometric analysis. From the moment a stage 4 or stage 5 NK cell is present in the culture, these cells are considered as the mature NK cell population (e.g. at least 1%, 5%, 10%, 15%, 20%, 30%, 40%, 50% or more of the cells in the culture have the respective phenotypic NK cell markers). Stage 4 and stage 5 NK cells are determined by a CD56⁺CD94⁺CD16⁻ and a CD56⁺CD94⁺CD16⁺ phenotype, respectively.

In a preferred embodiment, the “mature” NK cells are at least of stage 4, in particular stage 4 and stage 5, more in particular stage 5.

The method and medium used for the culturing (such as e.g. in step b or d) may be any suitable method and medium for culturing isolated HSCs. In particular said medium is IMDM medium (Iscove's Modified Dulbecco's Medium). Optionally said medium comprises about 1% to 20% serum (such as e.g. about 5%, 10%, 15%), in particular fetal calf serum or human AB serum. The medium of step d) may further contain a cytokine selected from the list consisting of: FLT3-L, SCF, IL-3, IL-7 and IL-15.

Furthermore, the culturing step d) may be a co-culturing step using any suitable co-culturing cell line or feeder cell line, such as for example an inactivated stromal cell line; more specifically EL08.1D2 cells (i.e. a murine fetal liver stromal cell line) or OP9 cells (i.e. a mouse bone marrow stromal cell line). We found that NK cells differentiated from HSC on the EL08.1D2 feeder cells express higher levels of KIRs and CD16 compared to NK cells differentiated on OP9 feeder cells, indicating increased NK cell maturation.

In a further aspect, the present invention also provides HSCs cells or NK cells which are characterized in that they are or have been transfected and/or transduced with at least one transcription factor selected from the list comprising: T-Box expressed in T cells (T-BET) and Eomesodermin (EOMES); or a combination thereof; in particular EOMES. Further enclosed are HSCs or NK cells transduced with a retroviral vector (e.g. the LZRS virus) containing the cDNA encoding T-BET and/or EOMES. In a particular embodiment, the invention provides HSCs transfected and/or transduced with EOMES, such as e.g. HSCs transduced with a retroviral vector (e.g. the LZRS virus) containing the cDNA encoding EOMES.

Finally, the present invention provides differentiated NK cells obtained using the methods of the present invention.

The invention also includes methods and uses of said NK cells in medical applications, such as e.g. immunotherapy and/or cancer treatment.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results.

1. Material and Methods

Isolation of CD34⁺ HSC from Umbilical Cord Blood

Umbilical cord blood (UCB) was obtained from the Cord Blood Bank, Ghent University Hospital, Ghent, Belgium. Cord blood usage in this study was approved by the Ethics Committee of the Faculty of Medicine and Health Sciences and informed consent was obtained in accordance with the Declaration of Helsinki. Mononuclear cells were obtained by Lymphoprep density gradient centrifugation. CD34⁺ HSC were subsequently enriched from the mononuclear cells using Magnetic Activated Cell Sorting (MACS; Direct CD34⁺ HSC MicroBead Kit, Miltenyi Biotech Leiden, The Netherlands) according to the manufacturer's guidelines. Purity of the CD34⁺ HSC was determined by labelling the cells with anti-CD34 antibody conjugated with phycoerythrine (PE). Purity of >90% was confirmed by a LSRII Flow Cytometer (BD Biosciences, San Jose, Calif., U.S.A). Freshly isolated CD34⁺ HSC were frozen in fetal calf serum (FCS)⁺10% DMSO and stored in liquid nitrogen until usage.

Retrovirus Production of Overexpression Vectors

Molecular Cloning of Overexpression Constructs

Human T-BET and EOMES cDNA was purchased from Source BioScience (Nottingham, UK; T-BET cDNA: IRATp970D0558D sequence is identical to NM_013351.1; EOMES cDNA: IRAKp961A1269Q sequence is identical to NM_001278182.1). Restriction sites for BamHI and Xho-I were added to the cDNA by PCR using Phusion® High Fidelity PCR (New England Biolabs Inc; Ipswich, Mass., U.S.A) with self-designed primers:

Fw-Tbet:
(SEQ. ID NO: 3)
AAGTTGGATCCACCATGGGCATCGTGGAGCCGGGTTG;
Rev-Tbet:
(SEQ. ID NO: 4)
AAAGTTCTCGAGTCAGTTGGGAAAATAGTTATAAAACTGTCCTTCAGCTT
CC;
Fw-Eomes:
(SEQ. ID NO: 5)
AAAGTTGGATCCACCATGCAGTTAGGGGAGCAGCTC;
Rev-Eomes:
(SEQ. ID NO: 6)
AAAGTTCTCGAGTTAGGGAGTTGTGTAAAAAGCATAATACCC.

Human ID2 and TOX cDNA were purchased from OriGene Technologies (Rockville, Md., U.S.A; ID2 cDNA: SC118791, sequence identical to NM_002166.4; TOX cDNA: SC114879, sequence identical to NM_014729.2). Restriction sites for BamHI, EcoRI and NgoMIV were added to the cDNA as described above. Self-designed primers:

| | Fw-ToxEcoRI: |
|---|---|
| (SEQ. ID NO: 7) | |
| | ATCTCAGAATTCAGTGAAATGGACGTAAGATTTTATCC |
| | Rev-ToxNgoMIV: |
| (SEQ. ID NO: 8) | |
| | AAAGTTGCCGGCTCAAGTAAGGTACAGTGCTTTGTCC |
| | Fw-Id2BamHI: |
| (SEQ. ID NO: 9) | |
| | CTATCAGGATCCGTCAGCATGAAAGCCTTCAGTC |
| | Rev-Id2NgoMIV: |
| (SEQ. ID NO: 10) | |
| | AAAGTTGCCGGCTCAGCCACACAGTGCTTTGC |

cDNA encoding human ETS-1 p51 or p27 was subcloned from the pLEXhEts1p51HAtag and pCDNA3hEts1p27 vector, respectively (kindly provided by L. A. Garrett-Sinha, State University of New York, Buffalo, N.Y., U.S.A., and [21]).

The cDNA of the different transcription factors was ligated into the LZRS-IRES-eGFP retroviral vector (original LZRS plasmid: T M Kinsella, G P Nolan (1996) [18]). The empty LZRS-IRES-eGFP vector was used as control. Viral vectors were sequenced (GATC Biotech, Ebersberg, Germany) to confirm correct DNA sequence of the constructs.

Retrovirus Production

The control, T-BET, EOMES, TOX, ID2 and ETS-1 retroviral constructs were transfected into Phoenix A cells using the Calcium Phosphate transfection kit (Invitrogen, Carlsbad, Calif., U.S.A) and maintained in Iscove's Modified Dulbecco's medium (IMDM) containing 10% FCS, 100 U/ml penicillin, 100 μg/ml streptomycin, 2 mM glutamine (Life technologies, Carlsbad, Calif., U.S.A) and 2 μg/ml puromycin. Retrovirus was harvested on day 2, day 6 and day 14 after transfection and stored at −80° C. until usage.

NK Cell Differentiation Culture In Vitro

Culture of EL08.1D2 Cells

The murine embryonic liver cell line EL08.1D2 was maintained in 50% Myelocult M5300 medium (Stem Cell Technologies, Grenoble, France), 35% α-MEM, 15% FCS, supplemented with 100 U/mL penicillin, 100 μg/mL streptomycin, 2 mM glutamine and 10 μM β-mercaptoethanol on 0.1% gelatin-coated plates at 33° C. EL08.1D2 cells were inactivated by adding 10 μg/ml mitomycin C to the culture medium during 2-3 hours. Cell proliferation of these cells is thereby completely blocked. Thereafter, cells were thoroughly rinsed before harvesting using trypsin-EDTA. Cells were plated at a density of 50,000 cells per well on a 0.1% gelatin-coated tissue culture-treated 24-well plate at least 24 h before adding HSC or before transfer of the differentiated NK cells/ILC3 on day 14 and day 21 of culture.

Retro Viral Transduction of HSC and NK Cell Differentiation

Isolated cord blood-derived CD34⁺ HSC were cultured in complete IMDM containing 10% FCS (all from Life Technologies) and supplemented with thrombopoietin (TPO) (20 ng/ml), stem cell factor (SCF) (100 ng/ml) (all from Peprotech) and FMS-like tyrosine kinase 3 ligand (FLT3-L) (100 ng/ml, R&D Systems) from day −4 to day −2. Subsequently, these cells were harvested, transferred to RetroNectin (Takara Bio, Saint-Germain-en-Laye, France)-coated plates and viral supernatant was added. Additional cytokines were added to keep the concentrations constant after virus addition. The plates were centrifuged at 950 g and 32° C. during 90 min. At day 0, lineage⁻(CD3/CD14/CD19/CD56) CD34⁺eGFP⁺ HSC were sorted using a FACS ARIA III cell sorter (BD Biosciences, San Jose, Calif., U.S.A.). Sorted HSC were co-cultured with mitomycin-treated EL08.1D2 cells in Dulbecco's modified Eagle medium plus Ham's F-12 medium (2:1 ratio), supplemented with 100 U/mL penicillin, 100 μg/mL streptomycin, 2 mM glutamine, 10 mM sodium pyruvate (all from Life Technologies), 20% of heat-inactivated human AB serum (Merck, Darmstadt, Germany), 24 μM β-mercaptoethanol, 20 μg/mL ascorbic acid and 50 ng/mL sodium selenite (all from Sigma-Aldrich). The following cytokines were added: IL-3 (5 ng/mL, first week only), IL-7 (20 ng/mL), IL-15 (10 ng/mL) (all from R&D Systems), SCF (20 ng/mL), and Flt3-L (10 ng/mL). Alternatively, to test the necessity of IL-15 in NK cell differentiation upon T-BET and EOMES transduction, IL-15 was not included in the cytokine mix. Culture medium was refreshed on day 7 by addition of the same volume of fresh medium with cytokines. At day 14 the non-adherent cells were harvested and transferred to new mitomycin-treated EL08.1D2 feeder cells.

Flow Cytometry

NK cell differentiation co-cultures were examined at different time points using flow cytometry (LSRII flow cytometer, BD Biosciences). Data were analyzed with FACSDiva Version 6.1.2 Software (BD Biosciences) and/or FlowJo_V10 (Ashland, Oreg., U.S.A).

Functional Assay

IFN-γ & TNF-α Production

For intracellular IFN-γ and TNF-α detection by flow cytometry, 10⁵ cells from day 21 T-BET and EOMES overexpression cultures, or from day 21 control transduced cells, were stimulated with 50 ng/ml phorbol myristate acetate (PMA) and 1 μg/ml ionomycin (both from Sigma Aldrich, Sant Louis, Mo., U.S.A); or with 10 ng/ml IL-12 (PeproTech, London, U.K.) and 10 ng/ml IL-18 (R&D Systems, MN, U.S.A.), with or without 10 ng/ml IL-15 (Miltenyi Biotec, Leiden, The Netherlands) for 24 h. For the last 4 h, brefeldin A (BD GolgiPlug, 1/1000, BD Biosciences) was added. Thereafter, NK cell marker surface staining was performed, followed by fixation and permeabilisation using the Cytofix/Cytoperm Kit (BD Biosciences) and IFN-γ/TNF-α staining. The presence of intracellular IFN-γ or TNF-α was analyzed by flow cytometry on the gated NK cells.

Cytotoxicity Assays

To determine cell specific killing, ⁵¹Chromium release assays were performed. Therefore, 10⁶ K562 target cells were labeled with 100 μCi Na₂⁵¹CrO₄ (Perkin Elmer, Waltham, Mass., U.S.A) for 1.5 h at 37° C. eGFP⁺CD45⁺CD11a⁺CD56⁺CD94⁺ NK cells were sorted from day 21 T-BET and EOMES overexpression cultures, or from control-transduced cultures, and were added in a serial dilution to 10^{3 51}Cr-labeled K562 cells per well in a V-bottomed 96-well plate. Effector cells were added to the targets cells in triplicate. After 4 h, the supernatant was harvested and radioactivity was measured using a Luminescence counter (Wallac Microbeta Trilux, Perkin Elmer). The percentage of specific lysis was calculated using the formula: [(experimental release−spontaneous release)/(maximal release−spontaneous release)]×100.

ADCC against Raji, a CD20-expressing human Burkitt's lymphoma cell line, was measured in triplicates using the ⁵¹Chromium release assay as described above. The target cells were added to the effector cells at an effector:target ratio of 1:1 in medium containing either 0 or 10 μg/ml Rituximab (anti-CD20 antibody) (Hoffmann-La Roche, Basel, Switzerland, kindly provided by the pharmacy of Ghent University Hospital, Belgium) and incubated for 4 h. Specific lysis was calculated using the formula as described above.

CD107a Degranulation Assay

For analysis of CD107a expression on the cell membrane, that is a measure of degranulation, 10⁵ cells from day 21 T-BET or EOMES overexpression cultures and from control transduced cells were added to 10⁵ K562 or Raji targets cells, with 0 or 10 μg/ml Rituximab, and co-cultured for 2 h. Thereafter, the cells were harvested and stained for NK cell surface markers and CD107a. CD107a degranulation in the gated NK cells was analyzed using flow cytometry.

Cytospins

For microscopic evaluation of the cell morphology, eGFP⁺CD45⁺CD11a⁺CD56⁺CD94⁺ NK cells were sorted from day 3 or day 7 T-BET and EOMES overexpression cultures, or from day 19 control-transduced cultures. Cytospins were made (Shandon Cytospin™ 4, Thermo Scientific, Cheshire, UK), Wright-Giemsa stained and microscopically evaluated. The percentage of cells containing cytotoxic granules was counted manually.

Library Prep and RNA Sequencing

After RNA extraction (RNeasy micro kit, Qiagen, Hilden, Germany), the concentration and quality of the total extracted RNA was checked by using the ‘Quant-it ribogreen RNA assay’ (Life Technologies, Grand Island, N.Y., U.S.A) and the RNA 6000 nano chip (Agilent Technologies, Santa Clara, Calif., U.S.A), respectively. Subsequently, 70 ng of RNA was used to perform an Illumina sequencing library preparation using the QuantSeq 3′ mRNA-Seq Library Prep Kits (Lexogen, Vienna, Austria) according to manufacturer's protocol. Libraries were quantified by qPCR, according to Illumina's protocol ‘Sequencing Library qPCR Quantification protocol guide’, version February 2011. A High sensitivity DNA chip (Agilent Technologies, Santa Clara, Calif., U.S.A.) was used to control the library's size distribution and quality. Sequencing was performed on a high throughput Illumina NextSeq 500 flow cell generating 75 bp single reads. Per sample, on average 5.3×10⁶±1.7×10⁵ reads were generated. First, these reads were trimmed using cutadapt version 1.11 to remove the “QuantSEQ FWD” adaptor sequence. The trimmed reads were mapped against the Homo sapiens GRCh38.90 reference genome using STAR version 2.5.3a. The RSEM software version 1.2.31 was used to generated the count tables.

To explore if the samples from different treatment groups clustered together and to detect outlier samples, a Principal Component Analysis (PCA) on rlog transformed counts was performed using the R statistical computing software. No outliers among the samples were detected. Differential gene expression analysis was performed using edgeR, whereby HSC upon T-BET or EOMES overexpression were compared to control HSC. Differential expressed genes were tested with edgeR exact Test. Genes with an FDR <0.05 were considered significantly differential.

GSEA was performed using the GSEA software tool v2.2.2 of the Broad Institute [19, 20]. The ‘GSEAPreranked’ module was run using standard parameters and 1000 permutations.

2. Results

Accelerated Human NK Cell Development Upon T-BET and EOMES Overexpression

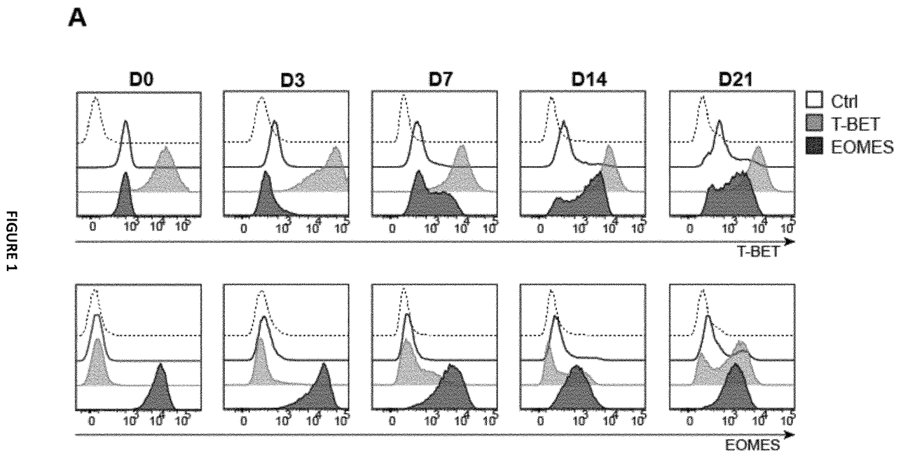

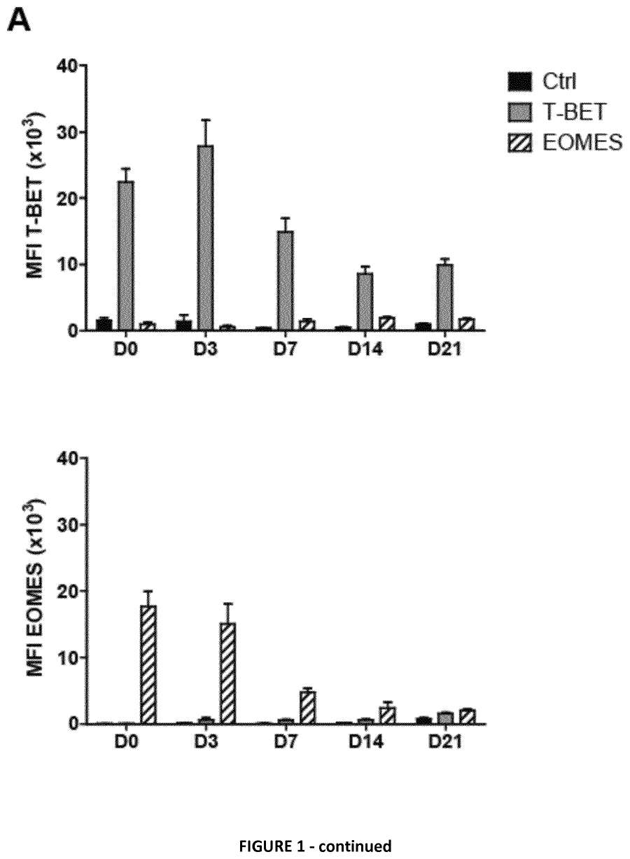

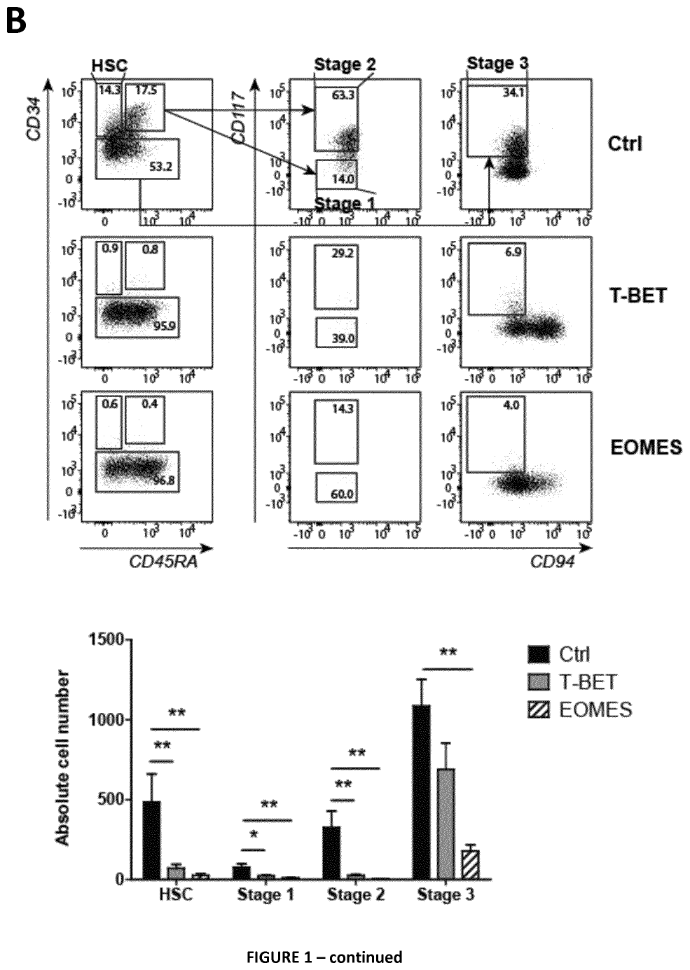

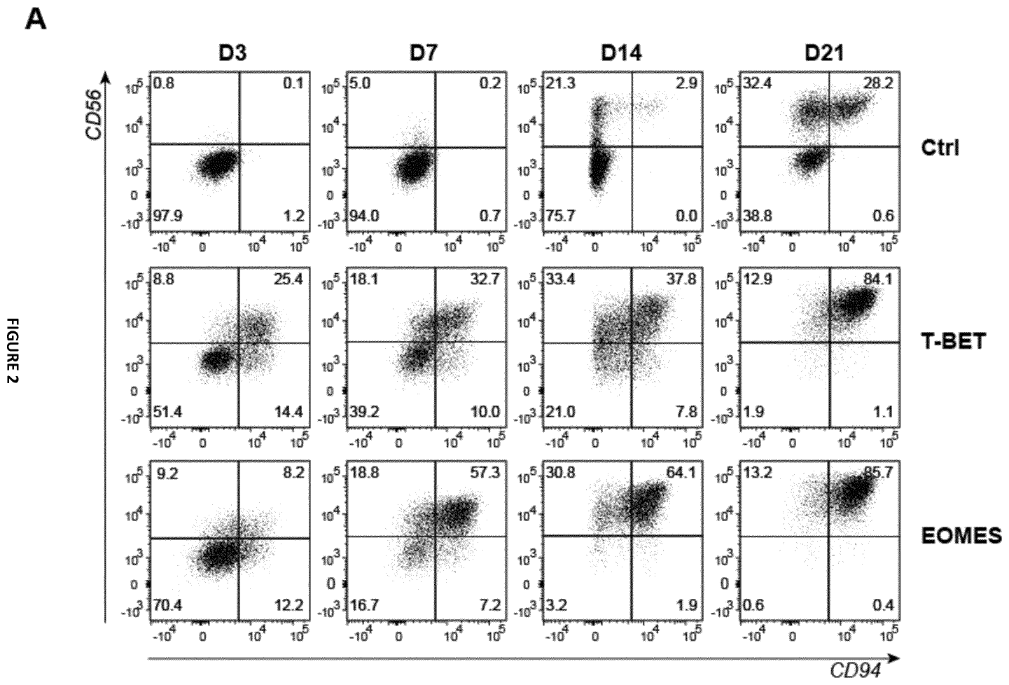

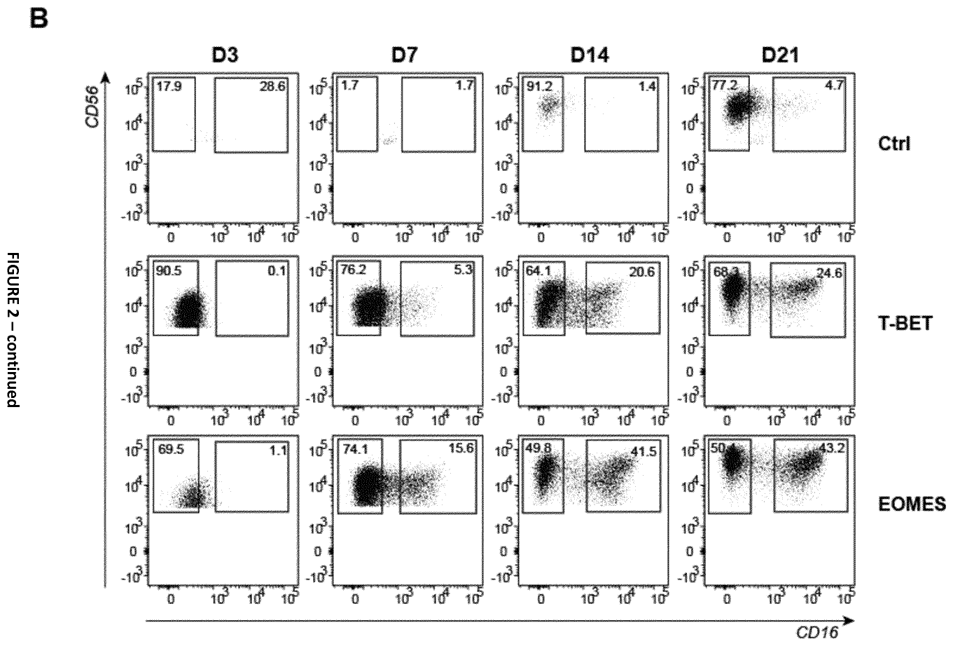

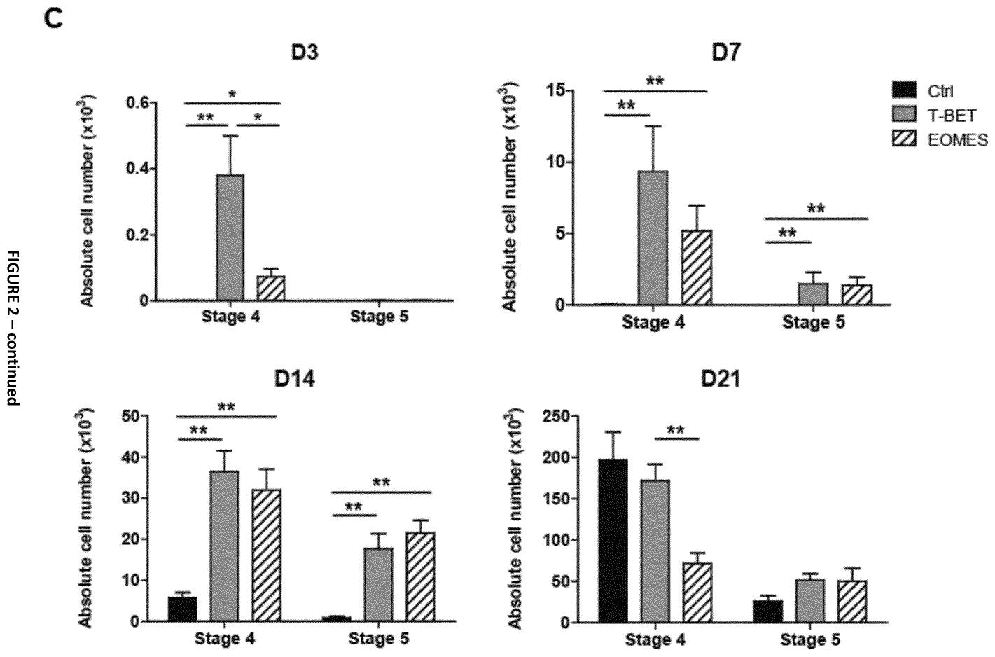

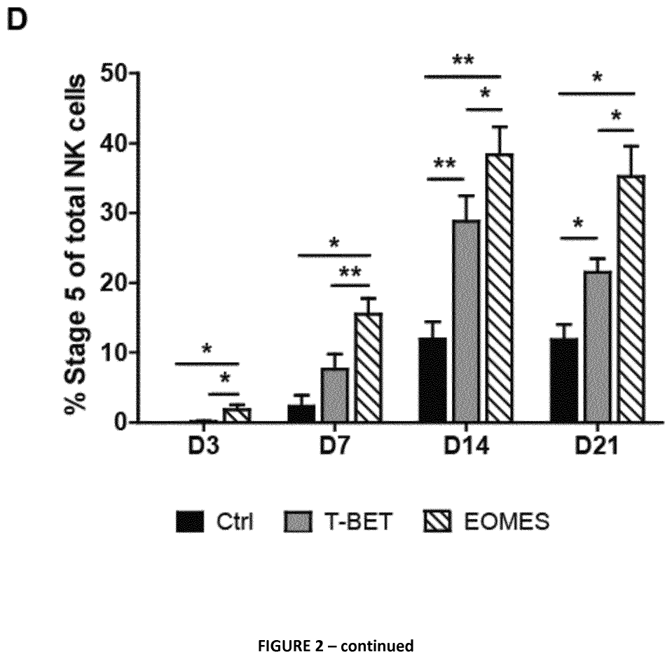

To investigate the regulatory role of transcription factors T-BET and EOMES in human NK cell development, overexpression constructs of both T-BET and EOMES were made, whereby human cDNA of T-BET or EOMES was cloned separately into the LZRS-IRES-eGFP retroviral vector. These overexpression constructs were transduced on day −2 in human umbilical cord blood-derived CD34⁺ HSC, in parallel to an empty control vector. At day 0, transduced HSC were sorted as lineage⁻(CD3/CD14/CD19/CD56) CD34⁺eGFP⁺ cells that were subsequently differentiated in the NK/ILC3 culture. From day 0, overexpression of T-BET and EOMES in eGFP⁺ cells was confirmed at regular time points at the protein level by flow cytometry, showing that overexpression was maintained throughout the culture period (FIG. 1a). On day 3, nearly no HSC, stage 1 and stage 2 NK cell progenitors were left in T-BET and EOMES overexpression cultures, whereas these populations were still clearly present in the control-transduced cultures. Also less stage 3 cells were found with T-BET and EOMES overexpression in comparison to control transduced cells (FIG. 1b). In sharp contrast, mature CD56⁺CD94⁺ NK cells, comprising stage 4 and stage 5 NK cells, were already present on day 3 of the T-BET and EOMES overexpression cultures, whereas NK cells only became detectable from day 14 in the control-transduced cells (FIG. 2a). On day 7, CD56⁺CD94⁺CD16⁺ NK cells, which are mature stage 5 NK cells, were present in the T-BET and EOMES overexpression cultures. In control cultures, stage 5 NK cells only appeared on day 21 (FIGS. 2b and 2c). Thus, both the percentages as well as the absolute numbers of stage 4 and stage 5 NK cells were significantly increased upon T-BET and EOMES overexpression at day 3, 7 and 14 (FIG. 2c-d)). With T-BET and EOMES overexpression, 21.5±4.3% and 35.2±9.7%, respectively, of total NK cells expressed CD16 on day 21 of culture, compared to 11.9±4.9% in control-transduced NK cells. Also the CD16 expression intensity was significantly higher in EOMES-overexpressing NK cells (mean fluorescence intensity (MFI) 6266±2709) compared to control NK cells (MFI 3984±1971) on day 21 of culture. The CD16 expression intensity of NK cells transduced with T-BET (MFI 4066±1457) did not differ significantly from control transduced NK cells.

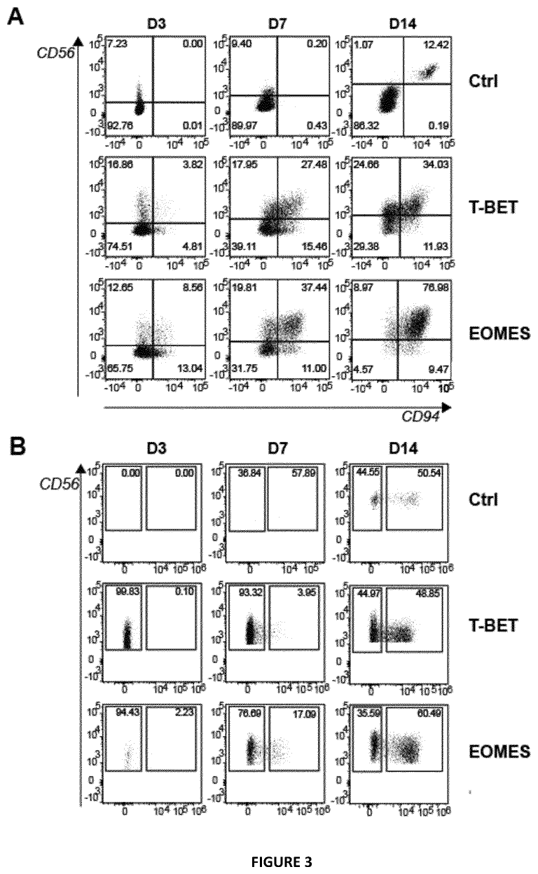

Currently, in vitro-generated NK cells used in NK cell immunotherapy are usually cultured in the absence of stromal feeder cells. To test whether accelerated NK cell differentiation upon T-BET or EOMES overexpression in HSC is also possible in a feeder-free system, transduced HSC were cultured in the NK cell/ILC3 differentiation culture, in the absence of EL08.1D2 feeder cells. The results show that, similar to NK cell cultures with stromal feeder cells, stage 4 NK cells were already present from day 3 of culture with both T-BET and EOMES overexpression cultures, whereas NK cells only became detectable on day 14 in control-cultures (FIGS. 3a and c). Furthermore, on day 7 of both the overexpression cultures mature stage 5 NK cells were present, whereas these mature NK cells only appeared on day 14 in the control-transduced cells (FIGS. 3b and c). The absolute cell number of mature stage 4 and stage 5 NK cells increased upon T-BET and EOMES overexpression at day 3, 7 and 14 (FIG. 3c), similar to the NK cell cultures with stromal feeder cells. Together, these data indicate that the presence of a feeder-layer does not influence the NK cell maturation upon T-BET and EOMES overexpression in HSC.

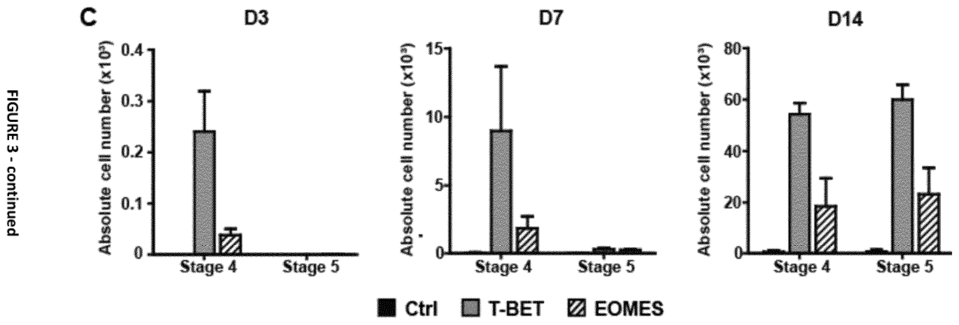

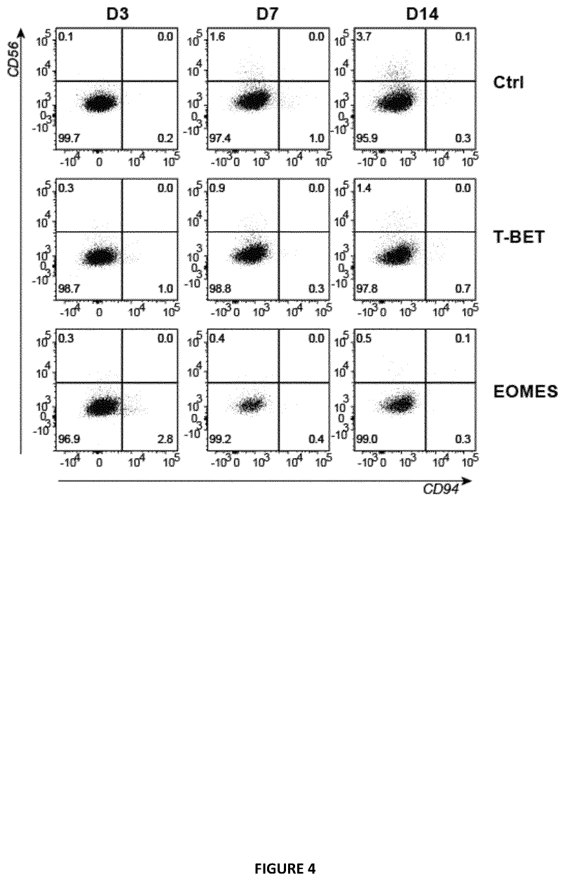

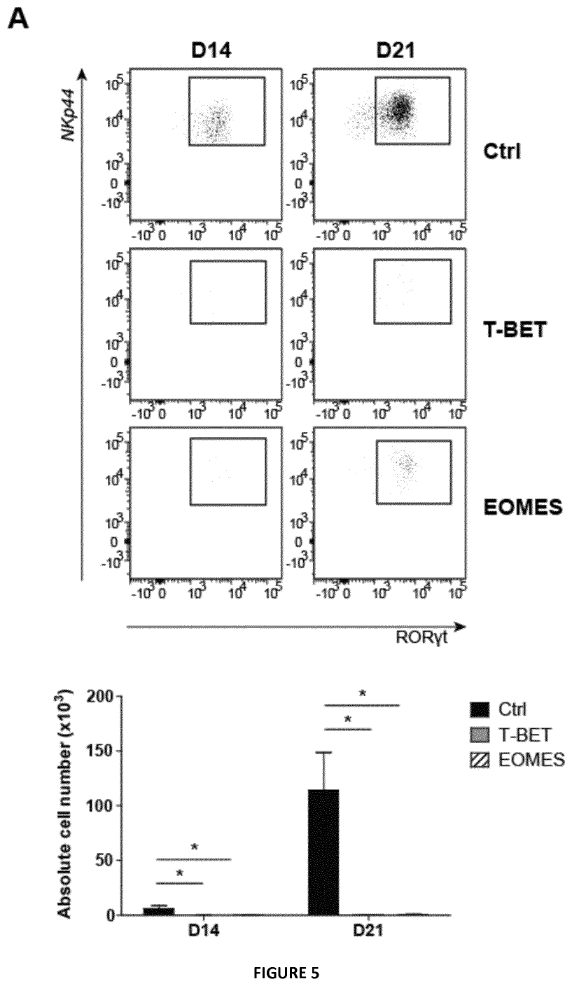

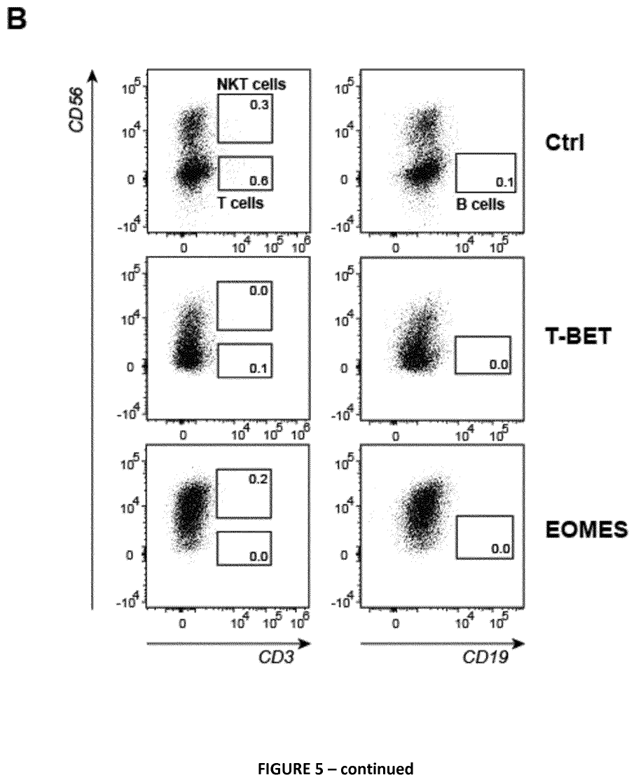

Because T-BET and EOMES overexpression in HSC led to extreme acceleration of NK cell differentiation, we reasoned that T-BET or EOMES overexpression might overrule the need for IL-15 in the culture medium. IL-15 is an important cytokine for NK cell development and differentiation through IL-2Rβ signaling in NK cell precursors. Results of cultures without IL-15 in the cytokine mix showed that, as control transduced HSC,-also T-BET- or EOMES-transduced HSC could not develop into NK cells on day 3. Even on day 14 of the culture period, no NK cells developed upon T-BET or EOMES overexpression, nor with control transduced cells (FIG. 4). This means that T-BET or EOMES overexpression in HSC does not overrule the necessity for IL-15 during NK cell differentiation. Moreover, with T-BET and EOMES overexpression in HSC, IL2R6 mRNA is upregulated in EOMES-transduced compared to control transduced HSC on day 0 (FIG. 9a). Cumulatively, this indicates that early precursors remain dependent on IL-15 for NK cell differentiation from HSCs upon T-BET or EOMES overexpression. In contrast to the accelerated and increased differentiation of NK cells, much less ILC3 developed upon T-BET and EOMES overexpression compared to the control (FIG. 5a), suggesting that ILC3 development is strongly inhibited by T-BET and EOMES overexpression. Furthermore, no B cells, T cells or NKT cells developed in the control or T-BET and EOMES overexpression cultures (FIG. 5b). Altogether, the differentiation of T-BET or EOMES transduced-HSC is thus completely skewed towards NK cell development, wherein differentiation towards stage 4 and stage 5 NK cells is drastically accelerated.

Early Arising NK Cells Upon T-BET and EOMES Overexpression Express a Mature NK Cell Phenotype

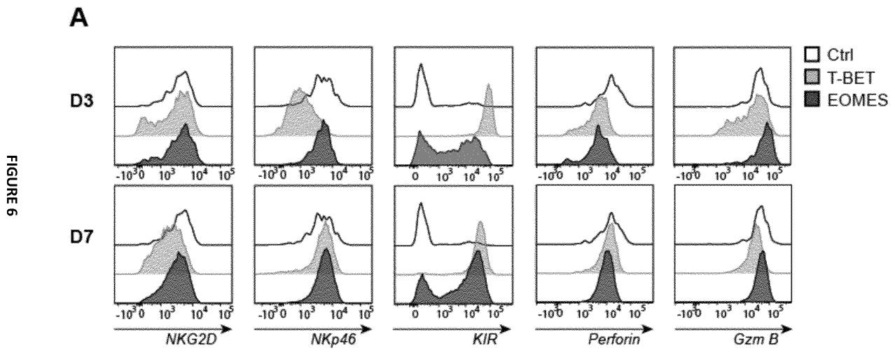

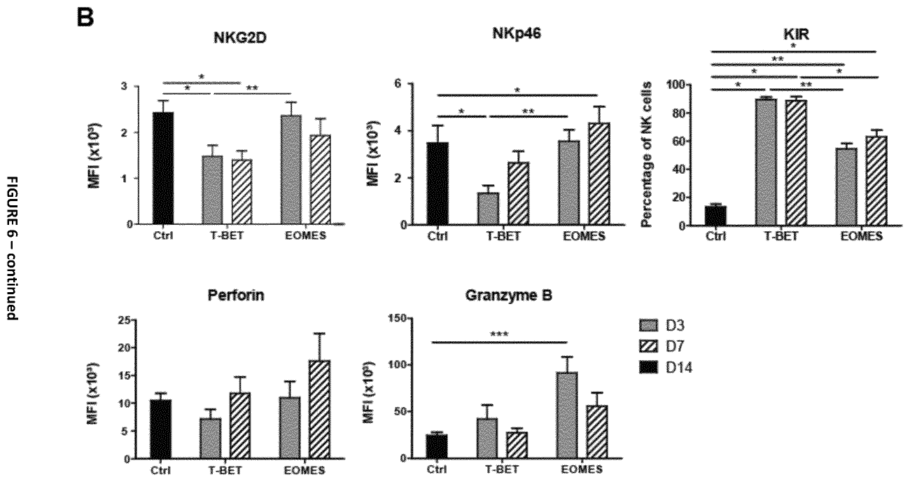

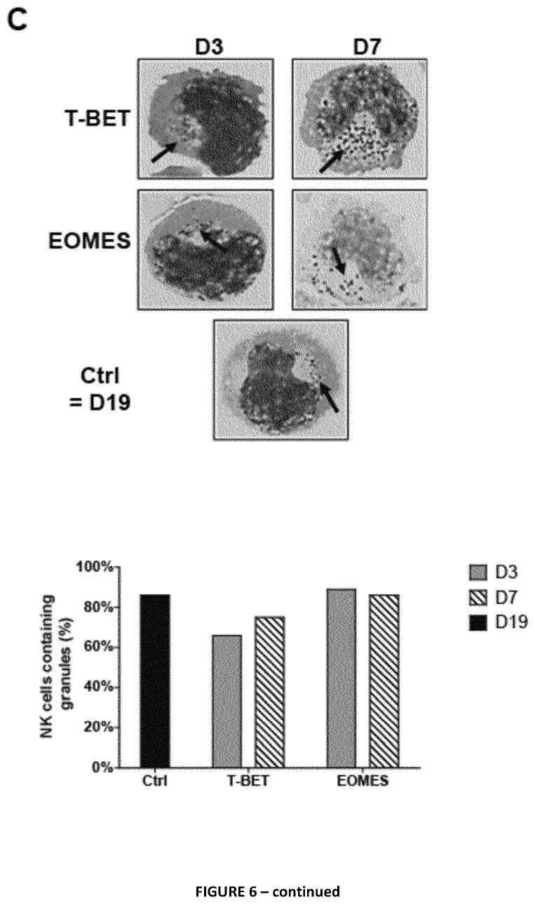

In order to further characterize the early arising NK cells upon T-BET and EOMES transduction of HSC, their phenotype was analyzed by flow cytometry using a panel of mature NK cell markers. As differentiating NK cells gradually express activating NK cell receptors, NKG2D and NKp46 expression was evaluated. NKG2D was expressed by NK cells from day 3 of the EOMES overexpression cultures, at a level comparable to day 14 control transduced NK cells (FIG. 6a-b). The NK cells upon T-BET overexpression also expressed NKG2D on day 3 of culture, although at a lower level compared to day 14 control transduced NK cells (FIG. 6a-b). Similar to NKG2D, NKp46 expression by NK cells with T-BET overexpression was delayed and only reached higher levels on day 7, while with EOMES overexpression NKp46 expression was expressed on day 3 at a comparable level to day 14 control transduced NK cells, and was expressed at higher levels on day 7 in comparison to control transduced NK cells (FIG. 6a-b). Next to activating NK cell receptors, mature NK cells also express killer-cell immunoglobulin-like receptors (KIRs). Evaluation of KIR expression by NK cells obtained at day 3 or day 7 upon T-BET and EOMES overexpression showed that KIR expression was extremely upregulated in comparison to day 14 control transduced NK cells (FIG. 6a-b). Finally, other markers that indicate functional NK cell maturation are cytoplasmic expression of perforin and granzyme B, which are both important cytotoxic mediators. On day 3, perforin and granzyme B were similarly expressed by NK cells in both the T-BET and EOMES overexpression cultures as compared to day 14 control transduced cells. At day 7, perforin expression in both T-BET and EOMES overexpressing NK cells tended to rise, but did not reach significant higher amounts in comparison to day 14 control NK cells (FIG. 6a-b). In contrast, granzyme B expression by NK cells with EOMES overexpression was significantly higher on day 3 of culture in comparison to day 14 control NK cells (FIG. 6a-b).

Perforin and granzyme B proteins are known to be contained in the cytotoxic granules of NK cells. We therefore performed microscopic analysis of sorted NK cells from day 3 and day 7 overexpression cultures, and from day 19 control cultures. The results show that NK cells from day 3 and day 7 T-BET and EOMES overexpression cultures had multiple cytotoxic granules in their cytoplasm, at equal numbers compared to day 19 control NK cells (FIG. 6c). In conclusion, T-BET or EOMES overexpression in HSC results in accelerated differentiation of human NK cells with a complete mature phenotype, which also contain cytotoxic granules in their cytoplasm.

The Early Arising NK Cells Upon T-BET and EOMES Overexpression are Functionally Mature

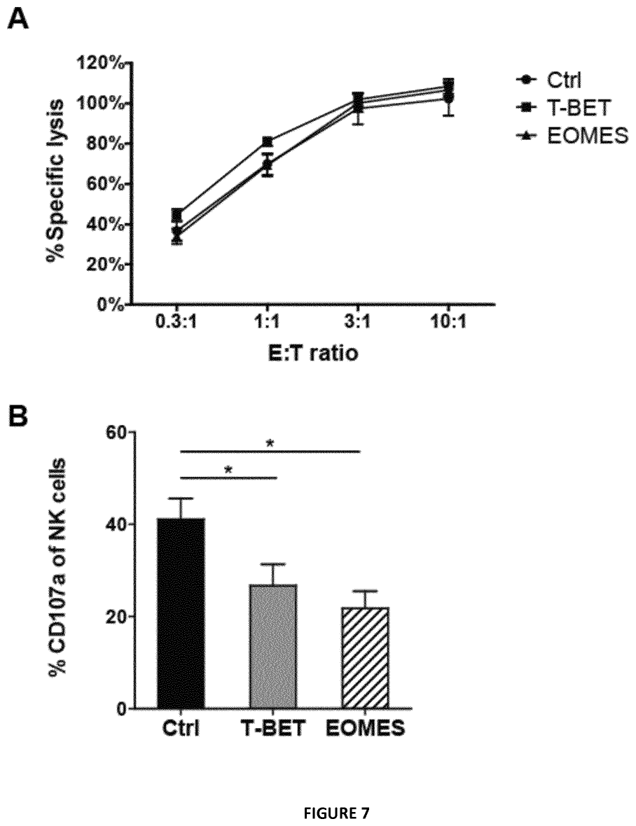

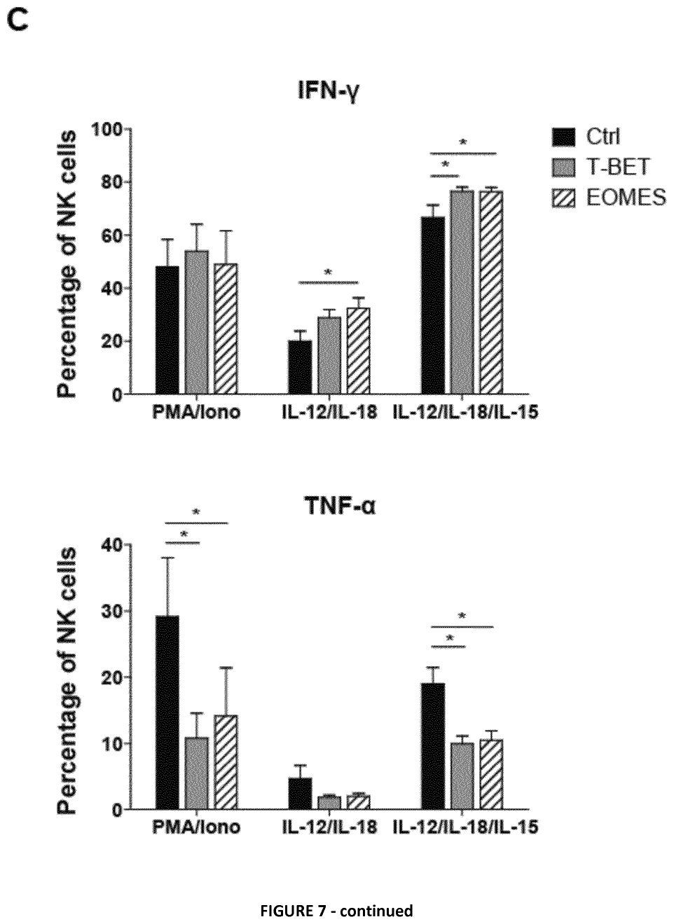

The most important function of mature NK cells is killing of malignant and virus-infected cells. Because the early arising NK cells, upon T-BET or EOMES overexpression in HSC, express both perforin and granzyme B and contain cytoplasmic granules, we reasoned that they also have cytotoxic potential. Therefore, cytotoxic assays were performed with the human NK cell susceptible K562 cell line as target cells. The results show that NK cells from day 21 T-BET and EOMES overexpression cultures mediated comparable cytotoxicity as day 21 control NK cells (FIG. 7a). To correlate the target cell lysis with cytotoxic granule degranulation, CD107a expression was determined, whereby day 21 NK cells from T-BET or EOMES overexpression cultures and control cultures were challenged with K562 cells in a 2 h degranulation assay. CD107a expression in both NK cells from T-BET and EOMES overexpression cultures was significantly lower in comparison to control transduced NK cells (FIG. 7b).

Another important function of mature NK cells is the production of pro-inflammatory cytokines, including IFN-γ and TNF-α, whereby they are able to influence other immune cells. Therefore, we stimulated day 21 NK cells from both overexpression and control cultures with PMA and ionomycin or with a combination of IL-12, IL-18 with or without IL-15. IFN-γ production of T-BET and EOMES overexpressing versus control NK cells was comparable after stimulation with PMA/ionomycin, and was higher upon IL-12/IL-18 or IL-12/IL-18/IL-15 stimulation (FIG. 7c). In contrast, TNF-α production of T-BET or EOMES overexpressing NK cells was significantly lower than control transduced NK cells for both the PMA/ionomycin and the IL-12/IL-18/IL-15 conditions (FIG. 7c). We can conclude that NK cells obtained upon T-BET and EOMES overexpression in HSC not only have a mature phenotype but are also functionally mature, both regarding cytotoxicity as well as IFN-γ production.

EOMES-Overexpressing NK Cells have Increased ADCC Activity

Antibody-dependent cellular cytotoxicity (ADCC) is a mechanism whereby the target cell is lysed due to the presence of bound antibodies to the target cell surface that cross-link activating Fc-receptors on the cell surface of the effector cells. CD16 (FcγRIII) is the main activating Fc-receptor widely expressed on NK cells and induces killing by ADCC. Significantly more CD16⁺ NK cells, both in percentages as well as in absolute cell numbers, were obtained in T-BET and EOMES overexpression cultures as compared to control cultures (FIG. 2c-d), and NK cells from the EOMES overexpression cultures also express CD16 at a higher intensity.

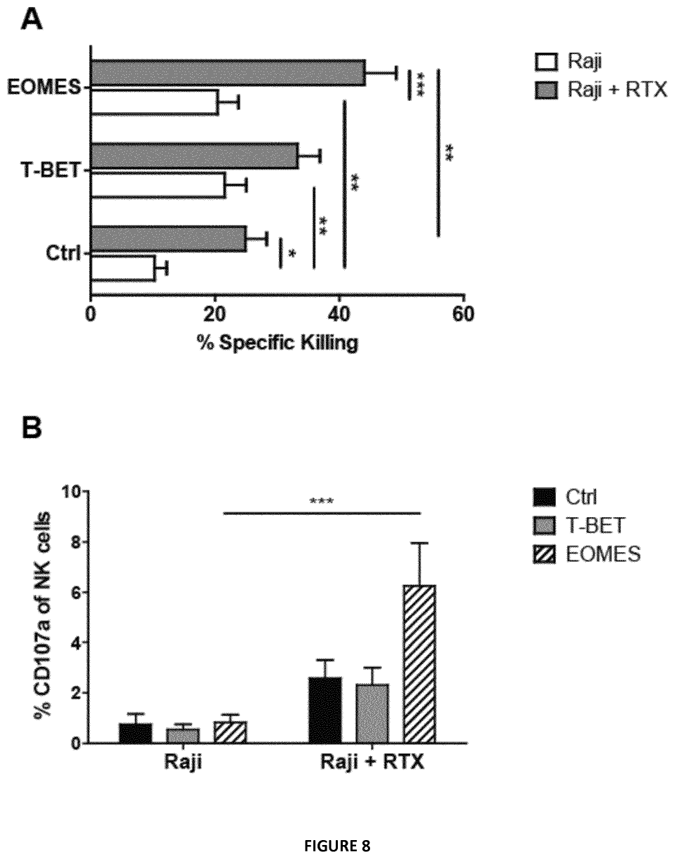

With regard to the therapeutic potential of the T-BET and/or EOMES overexpressing NK cells, we therefore tested their ADCC capacity. For this purpose, the CD20-expressing human Burkitt's lymphoma cell line Raji was used as target in the presence or absence of Rituximab (RTX), a humanized monoclonal anti-CD20 antibody that is used in cancer immunotherapy. The results show that both T-BET and EOMES overexpressing as well as control NK cells displayed ADCC, but the ADCC capacity of EOMES-overexpressing NK cells was significantly higher as compared to control NK cells (FIG. 8a). This was confirmed by CD107a degranulation analysis (FIG. 8b). The lower percentage of CD16⁺ NK cells and their lower CD16 expression intensity upon T-BET compared to EOMES overexpression can account for the lack of a stronger ADCC-response in T-BET overexpressing NK cells (FIG. 2d). Altogether, our results show that NK cells overexpressing EOMES display higher ADCC.

Transcriptome Profiling of T-BET and EOMES Overexpressing HSC Displays Activation of NK Cell Specific Genes.

In order to obtain a mechanistic insight into the accelerated differentiation and maturation of NK cells from T-BET or EOMES transduced versus control transduced HSC, their transcriptome was determined by RNA-sequencing. In T-BET and EOMES overexpressing HSC, 572 and 1427 differentially expressed genes (false discovery rate (FDR)<0.05), respectively, were identified.

HSC overexpressing T-BET or EOMES both showed higher expression of transcription factors that have a proven role in murine and/or human NK cell differentiation, including HELIOS (IKZF2), IRF8 and TOX. Higher expression of ETS-1 and RUNX2 was only present upon EOMES overexpression, while HOBIT (ZNF683) was only higher expressed in HSC overexpressing T-BET. In addition to transcription factors, also perforin (PRF1), granzyme B (GZMB) and IL2RB were higher expressed in EOMES-transduced HSC. As expected, CD34 expression was downregulated in both T-BET and EOMES overexpressing HSC (FIG. 9a).

TABLE 1
Differentially expressed genes highlighted
in the Volcano plots of FIG. 9

| | | | | |
|---|---|---|---|---|
| Gene | Gene | | log2fold- | |
| Name | Symbol | NCBI nr. | change | −Log10FDR |

T-BET vs Ctrl

| | | | | |
|---|---|---|---|---|
| HELIOS | IKZF2 | NM_016260.3 | 1.5531 | 1.10E−22 |
| IRF8 | IRF8 | NM_002163.4 | 1.0177 | 1.46E−07 |
| 2B4 | CD244 | NM_016382.4 | 1.0283 | 1.33E−05 |
| CD94 | KLRD1 | NM_002262.5 | 3.3074 | 3.96E−07 |
| Granzyme | GZMB | NM_004131.6 | 3.8296 | 1.35E−08 |
| B | | | | |
| HOBIT | ZNF683 | NM_001114759.2 | 6.0164 | 2.31E−12 |
| TOX | TOX | NM_014729.3 | 0.8980 | 2.65E−02 |
| CD34* | CD34 | NM_001025109.2 | −1.1166 | 6.45E−09 |

EOMES vs Ctrl

| | | | | |
|---|---|---|---|---|
| IRF8 | IRF8 | NM_002163.4 | 1.168523708 | 5.723E−16 |
| HELIOS | IKZF2 | NM_016260.3 | 1.442940756 | 9.961E−14 |
| Granzyme | GZMB | NM_004131.6 | 4.384453664 | 3.769E−13 |
| B | | | | |
| TOX | TOX | NM_014729.3 | 1.602786965 | 8.082E−12 |
| ETS-1 | ETS-1 | NM_001143820.2 | 1.746044341 | 1.513E−08 |
| RUNX2 | RUNX2 | NM_001024630.4 | 1.209127902 | 4.678E−06 |
| 2B4 | CD244 | NM_016382.4 | 0.815236302 | 0.0015388 |
| IL2RB | IL2RB | NM_000878.5 | 1.326326192 | 0.0214746 |
| Perforin | PRF1 | NM_001083116.3 | 1.193498197 | 0.0937772 |
| CD94 | KLRD1 | NM_002262.5 | 1.84207851 | 0.0752528 |
| CD34* | CD34 | NM_001025109.2 | −1.412926549 | 4.88E−09 |

*downregulated

TABLE 2
Top 10 downregulated genes

| | | | |
|---|---|---|---|
| Gene | | log2fold | |
| Symbol | NCBI nr. | change | −Log10FDR |

T-BET vs Ctrl

| | | | |
|---|---|---|---|
| SNAI1 | NM_005985.4 | −4.3285 | 4.42E−02 |
| OTUD3 | NM_015207.2 | −3.6426 | 2.62E−02 |
| WNT5B | NM_032642.3 | −3.5278 | 2.60E−02 |
| AHSP | NM_016633.4 | −3.4471 | 7.40E−03 |
| CPB1 | NM_001871.3 | −3.4147 | 7.21E−03 |
| TGM2 | NM_004613.4 | −3.1939 | 3.91E−03 |
| OSBP2 | NM_030758.4 | −3.0899 | 3.93E−02 |
| PF4 | NM_002619.4 | −2.7001 | 1.00E−08 |
| CDH1 | NM_004360.5 | −2.5450 | 1.72E−02 |
| F12 | NM_000505.3 | −2.2085 | 2.98E−02 |

EOMES vs Ctrl

| | | | |
|---|---|---|---|
| NMU | NM_006681.4 | −5.501 | 3.71E−03 |
| TRIM71 | NM_001039111.3 | −5.268 | 3.00E−03 |
| FLNC | NM_001458.4 | −4.829 | 7.33E−03 |
| SCN4B | NM_174934.3 | −4.715 | 2.24E−03 |
| NOG | NM_005450.6 | −4.670 | 5.27E−03 |
| AHSP | NM_016633.4 | −4.644 | 6.60E−04 |
| ENHO | NM_198573.3 | −4.043 | 4.99E−03 |
| TMEM158 | NM_015444.3 | −3.934 | 1.19E−02 |
| WNT5B | NM_032642.3 | −3.848 | 6.36E−03 |
| CEACAM4 | NM_001817.3 | −3.772 | 2.71E−05 |

FDR cutoff = 5.00E−02

TABLE 3
Top 10 upregulated genes

| | | | |
|---|---|---|---|
| Gene | | log2fold | |
| Symbol | NCBI nr. | change | padj |

T-BET vs Ctrl

| | | | |
|---|---|---|---|
| MS4A1 | NM_152866.2 | 7.428 | 2.86E−11 |
| ZNF683 | NM_001114759.2 | 6.016 | 2.31E−12 |
| INSM1 | NM_002196.3 | 5.890 | 2.32E−04 |
| GZMH | NM_033423.5 | 5.784 | 3.39E−09 |
| MNX1 | NM_005515.4 | 5.597 | 1.82E−03 |
| FGFBP2 | NM_031950.4 | 5.576 | 6.10E−05 |
| GYPB | NM_002100.6 | 5.442 | 8.63E−14 |
| TINAGL1 | NM_022164.3 | 4.758 | 2.98E−03 |
| SCN3A | NM_006922.4 | 4.523 | 1.16E−07 |
| NECAB1 | NM_022351.5 | 4.383 | 6.59E−14 |

EOMES vs Ctrl

| | | | |
|---|---|---|---|
| INSM1 | NM_002196.3 | 6.760 | 6.38E−08 |
| HP | NM_005143.5 | 6.744 | 2.80E−11 |
| GZMH | NM_033423.5 | 6.731 | 4.96E−13 |
| MYO5C | NM_018728.4 | 6.610 | 5.26E−08 |
| CA12 | NM_001218.5 | 6.454 | 1.86E−06 |
| LGALS2 | NM_006498.3 | 6.416 | 6.17E−06 |
| ABCA6 | NM_080284.3 | 6.294 | 1.32E−05 |
| MS4A1 | NM_152866.2 | 6.188 | 2.05E−06 |
| CHRNA3 | NM_000743.5 | 6.148 | 3.51E−05 |
| MNX1 | NM_005515.4 | 6.048 | 2.17E−05 |

FDR cutoff = 5.00E−02

The above tables provide an overview of the top 10 upregulated and downregulated genes in T-BET or EOMES transduced HSCs vs control. The genes listed therein are suitable for further differentiating the cells of the present invention from non-transduced/non-transfected or control-transduced/control-transfected ones.

Importantly, gene set enrichment analysis (GSEA) further revealed that T-BET or EOMES transduced HSC both have increased expression of a large set of mature CD56^{dim} NK cell specific genes (FIG. 9b). The transcriptome profiling results are compatible with the accelerated differentiation of T-BET or EOMES transduced HSC into NK cells.

Overexpression of ID2, TOX or ETS-1 in Human HSC does not Accelerate NK Cell Differentiation.

Several transcription factors have been shown to play crucial roles in NK cell lineage specification, differentiation and/or maturation. Both ETS proto-oncogene 1 (ETS-1) and Inhibitor of DNA binding 2 (ID2) have been shown to specify early stages of NK cell development and are key regulators of NK cell lineage specification in mice [7]. Moreover, ETS-1 deficiency in human HSC results in decreased NK cell differentiation in vitro, revealing a critical role for ETS-1 in human NK cell development [22]. The lack of mature NK cells was reported in mice that are deficient in Thymocyte Selection Associated High Mobility Group Box (TOX) [7]. This defect was also seen in human in vitro NK cell cultures, whereby the mature NK cell population decreases [15].

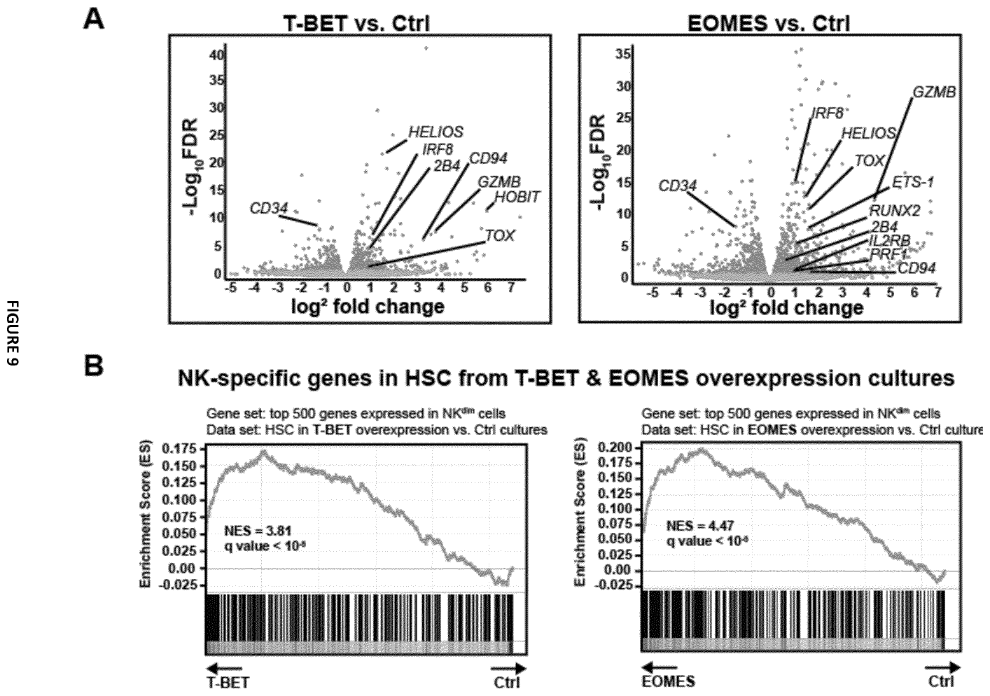

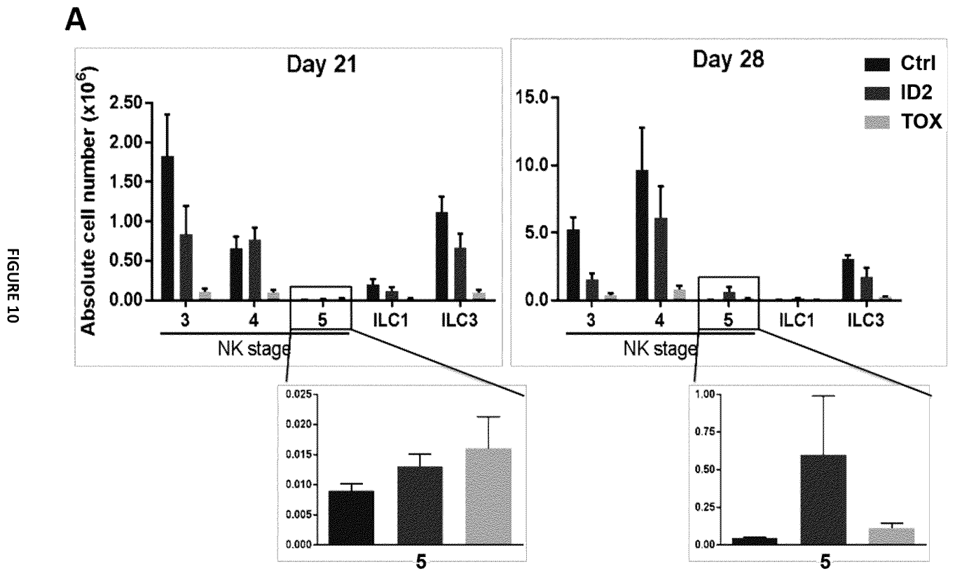

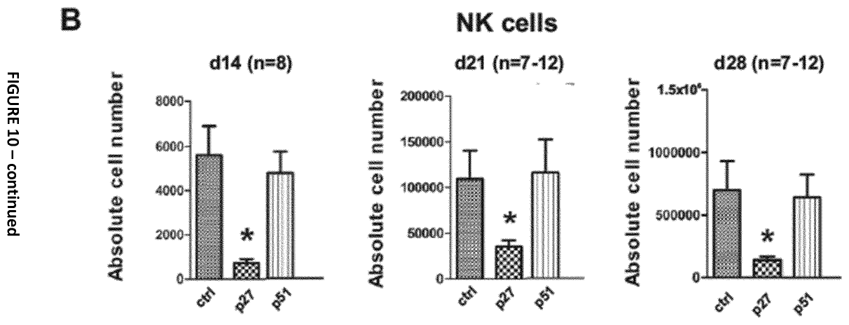

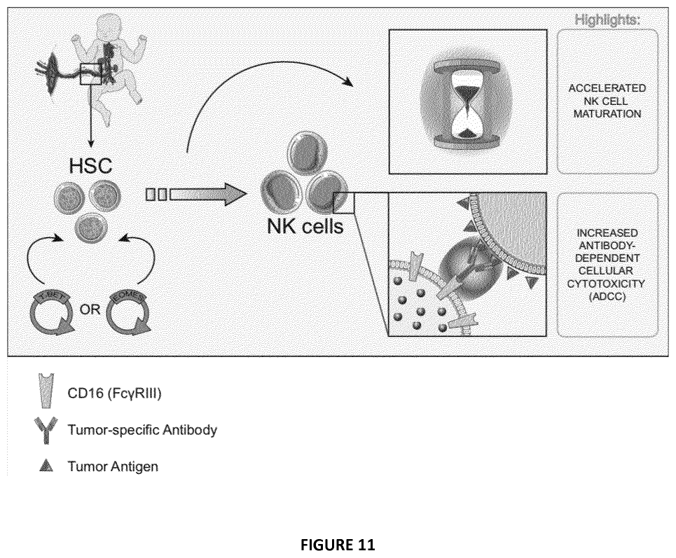

To analyze whether the accelerated NK cell differentiation and maturation observed with T-BET or EOMES overexpression also occurs with overexpression of other transcription factors involved in NK cell differentiation, we tested the effect of overexpression of ID2, TOX and ETS-1. ID2 overexpression in HSC did not result in significant differences in NK cell maturation in comparison to control transduced cells (FIG. 10a). Whereas TOX overexpression did not affect stage 5 NK cell differentiation, it did inhibit generation of stage 4 NK cells (FIG. 10a). Our findings are different from results of Yun S. et al. (15) that report increased NK cell differentiation upon TOX overexpression in human HSC. However, they only show increased NK cell percentages, whereas absolute NK cell numbers are not indicated. They also did not study CD16 expression on the generated NK cells.

We additionally overexpressed two isoforms of ETS-1 were overexpressed: p27, a dominant-negative isoform that inhibits signaling of endogenous ETS-1, and p51, the full-length isoform. Whereas p27 overexpression inhibited NK cell differentiation, showing a critical role for ETS-1 in this process, overexpression of the functionally active p51 isoform did not increase NK cell differentiation (FIG. 10b).

### REFERENCES

1. Spits H, et al. (2013). Nature reviews. immunology 13: 145-149.
2. Hazenberg M D, Spits H. (2014). Blood 124 (5): 700-709.
3. Caligiuri M A. (2008). Blood 112: 461-469.
4. Vivier E, et al. (2008). Nature immunology 9(5): 503-510.
5. Freud A G, et al. (2006). The journal of experimental medicine 203(4): 1033-1043
6. Yu J, et al. (2013). Trends in immunology 34(12): 573-582.
7. Goh W. & Huntington N D. (2017). Frontiers in immunology 8: 130.
8. Simonetta F, et al. (2016). Frontiers in immunology 7:241
9. Townsend M J, et al. (2004). Immunity 20: 477-494
10. Gordon S M, et al. (2012). Immunity 35: 55-67.
11. Pikovskaya O, et al. (2016). The journal of immunology 196: 1449-1454.
12. Rankin L C, et al. (2013). Nature immunology 14: 389-395.
13. Gill S, et al. (2012). Blood 119(24): 5758-5768.
14. Simonetta F, et al. (2015). The journal of immunology 195: 4712-4720
15. Yun S, et al. (2011) Immunology Letters 136:29-36
16. Vong Q P, et al (2014) Blood. 124:3905-13
17. Gacerez A T, Sentman C L. (2018). Cancer Gene Therapy 25:117-128
18. Kinsella T M, Nolan G P (1996) Human Gene Therapy 7:1405-1413
19. Subramanian A, et al. (2005). PNAS 102 (43): 15545-15550
20. Lindgren M, et al (2003). Nature Genetics volume 34: 267-273
21. Laitem, C., et al. (2009). Oncogene 28 (20): 2087-99.
22. Sylvie T, et al (2019). Submitted

1. A method of differentiating hematopoietic stem cells (HSC) into mature natural killer (NK) cells, said method comprising the steps of:
a) providing isolated HSCs;
b) culturing said cells of step a) in medium containing thrombopoietin (TPO), stem cell factor (SCF) and FMS-like tyrosine kinase 3 ligand (FLT3-L);
c) transfecting and/or transducing said cells of step b) with at least one transcription factor selected from the list comprising: T-Box expressed in T cells (T-BET) or Eomesodermin (EOMES); or a combination thereof;
d) culturing the cells obtained from step c) in a medium containing at least one cytokine selected from the list comprising IL-2 or IL-15; preferably IL-15;
whereby said mature NK cells are obtainable from day 3 after the start of step d).

2. The method according to claim 1, wherein the CD16 expression of said mature NK cells is increased compared to non-transfected or non-transduced control cells, or to control transfected or control transduced cells.

3. The method according to claim 1, wherein said mature NK cells are at least of stage 4.

4. The method according to claim 1, wherein said medium of step b) is complete Iscove's Modified Dulbecco's Medium (IMDM medium) comprising serum.

5. The method according to claim 1, wherein said TPO is present at a concentration from about 1 ng/ml to about 100 ng/ml.

6. The method according to claim 1, wherein said SCF is present at a concentration from about 5 ng/ml to about 500 ng/ml.

7. The method according to claim 1, wherein said FLT3-L is present at a concentration from about 5 ng/ml to about 500 ng/ml.

8. The method according to claim 1, wherein said medium of step d) further comprises a cytokine selected from the list comprising FLT3-L, SCF, IL-3 or IL-7.

9. The method according to claim 1, wherein said IL-2 and/or IL-15 is present at a concentration from about 0.5 ng/ml to about 50 ng/ml.

10. The method according to claim 1, wherein step d) is a co-culturing step using an inactivated stromal cell line; such as using EL08.1D2 cells or OP9 cells.

11. The method according to claim 1, wherein in step c) said cells are transduced with a retroviral vector comprising a nucleic acid encoding said at least one transcription factor.

12. A hematopoietic stem cell (HSC) transfected and/or transduced with T-Box expressed in T cells (T-BET), Eomesodermin (EOMES), or a combination of Eomesodermin (EOMES) and T-Box expressed in T cells (T-BET).

13. A differentiated NK cell obtained using the method according to claim 1.

14. The differentiated NK cell according to claim 13, whereby CD16 expression of said NK cells is increased compared to non-transfected or non-transduced control cells, or to control transfected or control transduced cells.

15. A method of inducing antibody-dependent cellular cytotoxicity in a subject having cancer, the method comprising use of a differentiated NK cell according to claim 13.

16. A method of inducing antibody-dependent cellular cytotoxicity in a subject having cancer, the method comprising use of the transfected and/or transduced HSC according to claim 12.
